SEQUENCE LISTING (13 sequences)

SEQ ID NO: 1 - DNA, 2593 bp, Homo sapiens
tgtcccagcctcctttgcgggtaaacagacatggccggcgaaggagatcagcaggacgctgcgcacaacatgggcaaccacctgccgctcctgcctgagagtgaggaagaagatgaaatggaagttgaagaccaggatagtaaagaagccaaaaaaccaaacatcataaattttgacaccagtctgccgacatcacatacatacctaggtgctgatatggaagaatttcatggcaggactttgcacgatgacgacagctgtcaggtgattccagttcttccacaagtgatgatgatcctgattcccggacagacattacctcttcagctttttcaccctcaagaagtcagtatggtgcggaatttaattcagaaagatagaacctttgctgttcttgcatacagcaatgtacaggaaagggaagcacagtttggaacaacagcagagatatatgcctatcgagaagaacaggattttggaattgagatagtgaaagtgaaagcaattggaagacaaaggttcaaagtccttgagctaagaacacagtcagatggaatccagcaagctaaagtgcaaattcttcccgaatgtgtgttgccttcaaccatgtctgcagttcaattagaatccctcaataagtgccagatatttccttcaaaacctgtctcaagagaagaccaatgttcatataaatggtggcagaaataccagaagagaaagtttcattgtgcaaatctaacttcatggcctcgctggctgtattccttatatgatgctgagaccttaatggacagaatcaagaaacagctacgtgaatgggatgaaaatctaaaagatgattctcttccttcaaatccaatagatttttcttacagagtagctgcttgtcttcctattgatgatgtattgagaattcagctccttaaaattggcagtgctatccagcgacttcgctgtgaattagacattatgaataaatgtacttccctttgctgtaaacaatgtcaagaaacagaaataacaaccaaaaatgaaatattcagtttatccttatgtgggccgatggcagcttatgtgaatcctcatggatatgtgcatgagacacttactgtgtataaggcttgcaacttgaatctgataggccggccttctacagaacacagctggtttcctgggtatgcctggactgttgcccagtgtaagatctgtgcaagccatattggatggaagtttacggccaccaaaaaagacatgtcacctcaaaaattttggggcttaacgcgatctgctctgttgcccacgatcccagacactgaagatgaaataagtccagacaaagtaatactttgcttgtaaacagatgtgatagagataaagttatctaacaaattggttatattctaagatctgctttggaaattattgcctctgatacatacctaagtaaacataacattaatacctaagtaaacataacattacttggagggttgcagtttctaagtgaaactgtatttgaaacttttaagtatactttaggaaacaagcatgaacggcagtctagaataccagaaacatctacttgggtagcttggtgccattatcctgtggaatctgatatgtctggtagcatgtcattgatgggacatgaagacatctttggaaatgatgagattatttcctgtgttaaaaaaaaaaaaaatcttaaattcctacaatgtgaaactgaaactaataatttgatcctgatgtatgggacagcgtatctgtaccagtgctctaaataacaaaagctagggtgacaagtacatgttccttttggaaagaagcaaggcaatgtatattaattattctaaaagggctttgttcctttccattttctttaacttctctgagatactgatttgtaaattttgaaaattagttaaaatatgcagttttttgagcccacgaatagttgtcatttcctttatgtgcctgttagtaaaaagtagtattgtgtatttgctcagtatctgaactataagcccatttatactgttccatacaaaagctatttttcaaaaattaatttgaaccaaaactactactatagggaaaagatgccaaaacatgtcccctcacccaggctaaacttgatactgtattattttgttcaatgtaaattgaagaaaatctgtaagtaagtaaaccttaagtgtgaaactaaacatgttctttgttcaaataatgtaaaatatctactcataattttaaagttctaaaaaggctgctttgccacctacctctttgcttatattcaagggtttagtgggtccttccttccacatttgtatgataactttcgttttattggtagtcctttgctactttaaaactaatcaggttaaatgtttactcaaatgtagtaatagtatgaggccaaaatacatgccttgaaggtggagtgaaatcagttgagtgtcacactgcatatttatgtaaatagagtaaatgaaaattcaagaatacttccaatcaggatatgcccctgccttattttttctaaggtctacttttactactttaataattttgggtttgtgtgaaaaaa

SEQ ID NO: 2 - DNA, 2187 bp, Homo sapiens
agacatggccggcgaaggagatcagcaggacgctgcgcacaacatgggcaaccacctgccgctcctgcctgcagagagtgaggaagaagatgaaatggaagttgaagaccaggatagtaaagaagccaaaaaaccaaacatcataaattttgacaccagtctgccgacatcacatacatacctaggtgctgatatggaagaatttcatggcaggactttgcacgatgacgacagctgtcaggtgattccagttcttccacaagtgatgatgatcctgattcccggacagacattacctcttcagctttttcaccctcaagaagtcagtatggtgcggaatttaattcagaaagatagaacctttgctgttcttgcatacagcaatgtacaggaaagggaagcacagtttggaacaacagcagagatatatgcctatcgagaagaacaggattttggaattgagatagtgaaagtgaaagcaattggaagacaaaggttcaaagtccttgagctaagaacacagtcagatggaatccagcaagctaaagtgcaaattcttcccgaatgtgtgttgccttcaaccatgtctgcagttcaattagaatccctcaataagtgccagatatttccttcaaaacctgtctcaagagaagaccaatgttcatataaatggtggcagaaataccagaagagaaagtttcattgtgcaaatctaacttcatggcctcgctggctgtattccttatatgatgctgagaccttaatggacagaatcaagaaacagctacgtgaatgggatgaaaatctaaaagatgattctcttccttcaaatccaatagatttttcttacagagtagctgcttgtcttcctattgatgatgtattgagaattcagctccttaaaattggcagtgctatccagcgacttcgctgtgaattagacattatgaataaatgtacttccctttgctgtaaacaatgtcaagaaacagaaataacaaccaaaaatgaaatattcagtttatccttatgtgggccgatggcagcttatgtgaatcctcatggatatgtgcatgagacacttactgtgtataaggcttgcaacttgaatctgataggccggccttctacagaacacagctggtttcctgggtatgcctggactgttgcccagtgtaagatctgtgcaagccatattggatggaagtttacggccaccaaaaaagacatgtcacctcaaaaattttggggcttaacgcgatctgctctgttgcccacgatcccagacactgaagatgaaataagtccagacaaagtaatactttgcttgtaaacagatgtgatagagataaagttatctaacaaattggttatattctaagatctgctttggaaattattgcctctgatacatacctaagtaaacataacattaatacctaagtaaacataacattacttggagggttgcagtttctaagtgaaactgtatttgaaacttttaagtatactttaggaaacaagcatgaacggcagtctagaataccagaaacatctacttgggtagcttggtgccattatcctgtggaatctgatatgtctggtagcatgtcattgatgggacatgaagacatctttggaaatgatgagattatttcctgtgttaaaaaaaaaaaaaatcttaaattcctacaatgtgaaactgaaactaataatttgatcctgatgtatgggacagcgtatctgtaccagtgctctaaataacaaaagctagggtgacaagtacatgttccttttggaaagaagcaaggcaatgtatattaattattctaaaagggctttgttcctttccattttctttaacttctctgagatactgatttgtaaattttgaaaattagttaaaatatgcagttttttgagcccacgaatagttgtcatttcctttatgtgcctgttagtaaaaagtagtattgtgtatttgctcagtatctgaactataagcccatttatactgttccatacaaaagctatttttcaaaaattaatttgaaccaaaactactactatagggaaaagatgccaaaacatgtcccctcacccagactaaacttgatactgtattattttgttcaatgtaaattgaagaaaatctgtaagtaagtaaaccttaagtgtgaaactaaa

SEQ ID NO: 3 - DNA, 2658 bp, Homo sapiens
aaactcccggaagcggcatccacacctgatggtgtgactcggccgacgcgagcgccgcgcttcgcttcagctgctagctggcccaagggaggcgaccgcggagggtggcgaggggcggccaggacccgcagccccggggccgggccggtccggaccgccagggagggcaggtcagtgggcagatcgcgtccgcgggattcaatctctgcccgctctgataacagtccttttccctggcgctcacttcgtgcctggcacccggctgggcgcctcaagaccgttgtctcttcgatcgcttctttggacttggcgaccatttcagagatgtcttccagaagtaccaaagatttaattaaaagtaagtggggatcgaagcctagtaactccaaatccgaaactacattagaaaaattaaagggagaaattgcacacttaaagacatcagtggatgaaatcacaagtgggaaaggaaagctgactgataaagagagacacagacttttggagaaaattcgagtccttgaggctgagaaggagaagaatgcttatcaactcacagagaaggacaaagaaatacagcgactgagagaccaactgaaggccagatatagtactaccacattgcttgaacagctggaagagacaacgagagaaggagaaaggagggagcaggtgttgaaagccttatctgaagagaaagacgtattgaaacaacagttgtctgctgcaacctcacgaattgctgaacttgaaagcaaaaccaatacactccgtttatcacagactgtggctccaaactgcttcaactcatcaataaataatattcatgaaatggaaatacagctgaaagatgctctggagaaaaatcagcagtggctcgtgtatgatcagcagcgggaagtctatgtaaaaggacttttagcaaagatctttgagttggaaaagaaaacggaaacagctgctcattcactcccacagcagacaaaaaagcctgaatcagaaggttatcttcaagaagagaagcagaaatgttacaacgatctcttggcaagtgcaaaaaaagatcttgaggttgaacgacaaaccataactcagctgagttttgaactgagtgaatttcgaagaaaatatgaagaaacccaaaaagaagttcacaatttaaatcagctgttgtattcacaaagaagggcagatgtgcaacatctggaagatgataggcataaaacagagaagatacaaaaactcagggaagagaatgatattgctaggggaaaacttgaagaagagaagaagagatccgaagagctcttatctcaggtccagtttctttacacatctctgctaaagcagcaagaagaacaaacaagggtagctctgttggaacaacagatgcaggcatgtactttagactttgaaaatgaaaaactcgaccgtcaacatgtgcagcatcaattgcatgtaattcttaaggagctccgaaaagcaagaaatcaaataacacagttggaatccttgaaacagcttcatgagtttgccatcacagagccattagtcactttccaaggagagactgaaaacagagaaaaagttgccgcctcaccaaaaagtcccactgctgcactcaatgaaagcctggtggaatgtcccaagtgcaatatacagtatccagccactgagcatcgcgatctgcttgtccatgtggaatactgttcaaagtagcaaaataagtatttgttttgatattaaaagattcaatactgtattttctgttagcttgtgggcattttgaattatatatttcacattttgcataaaactgcctatctacctttgacactccagcatgctagtgaatcatgtatcttttaggctgctgtgcatttctcttggcagtgatacctccctgacatggttcatcatcaggctgcaatgacagaatgtggtgagcagcgtctactgagactactaacattttgcactgtcaaaatacttggtgaggaaaagatagctcaggttattgctaatgggttaatgcaccagcaagcaaaatattttatgttttgggggttttgaaaaatcaaagataattaaccaaggatcttaactgtgttcgcattttttatccaagcacttagaaaacctacaatcctaattttgatgtccattgttaagaggtggtgatagatactattttttttttcatattgtatagcggttattagaaaagttggggattttcttgatctttattgctgcttaccattgaaacttaacccagctgtgttccccaactctgttctgcgcacgaaacagtatctgtttgaggcataatcttaagtggccacacacaatgttttctcttatgttatctggcagtaactgtaacttgaattacattagcacattctgcttagctaaaattgttaaaataaactttaataaacccatgtagccctctcatttgattgacagtattttagttatttttggcattcttaaagctgggcaatgtaatgatcagatctttgtttgtctgaacaggtatttttatacatgctttttgtaaaccaaaaacttttaaatttcttcaggttttctaacatgcttaccactgggctactgtaaatgagaaaagaataaaattatttaatgttttaa

SEQ ID NO: 4 - DNA, 2516 bp, Homo sapiens
aaactcccggaagcggcatccacacctgatggtgtgactcggccgacgcgagcgccgcgcttcgcttcagctgctagctggcccaagggaggcgaccgcggagggtggcgaggggcggccaggacccgcagccccggggccgggccggtccggaccgccagggagggcagaccatttcagagatgtcttccagaagtaccaaagatttaattaaaagtaagtggggatcgaagcctagtaactccaaatccgaaactacattagaaaaattaaagggagaaattgcacacttaaagacatcagtggatgaaatcacaagtgggaaaggaaagctgactgataaagagagacacagacttttggagaaaattcgagtccttgaggctgagaaggagaagaatgcttatcaactcacagagaaggacaaagaaatacagcgactgagagaccaactgaaggccagatatagtactaccacattgcttgaacagctggaagagacaacgagagaaggagaaaggagggagcaggtgttgaaagccttatctgaagagaaagacgtattgaaacaacagttgtctgctgcaacctcacgaattgctgaacttgaaagcaaaaccaatacactccgtttatcacagactgtggctccaaactgcttcaactcatcaataaataatattcatgaaatggaaatacagctgaaagatgctctggagaaaaatcagcagtggctcgtgtatgatcagcagcgggaagtctatgtaaaaggacttttagcaaagatctttgagttggaaaagaaaacggaaacagctgctcattcactcccacagcagacaaaaaagcctgaatcagaaggttatcttcaagaagagaagcagaaatgttacaacgatctcttggcaagtgcaaaaaaagatcttgaggttgaacgacaaaccataactcagctgagttttgaactgagtgaatttcgaagaaaatatgaagaaacccaaaaagaagttcacaatttaaatcagctgttgtattcacaaagaagggcagatgtgcaacatctggaagatgataggcataaaacagagaagatacaaaaactcagggaagagaatgatattgctaggggaaaacttgaagaagagaagaagagatccgaagagctcttatctcaggtccagtttctttacacatctctgctaaagcagcaagaagaacaaacaagggtagctctgttggaacaacagatgcaggcatgtactttagactttgaaaatgaaaaactcgaccgtcaacatgtgcagcatcaattgcatgtaattcttaaggagctccgaaaagcaagaaatcaaataacacagttggaatccttgaaacagcttcatgagtttgccatcacagagccattagtcactttccaaggagagactgaaaacagagaaaaagttgccgcctcaccaaaaagtcccactgctgcactcaatgaaagcctggtggaatgtcccaagtgcaatatacagtatccagccactgagcatcgcgatctgcttgtccatgtggaatactgttcaaagtagcaaaataagtatttgttttgatattaaaagattcaatactgtattttctgttagcttgtgggcattttgaattatatatttcacattttgcataaaactgcctatctacctttgacactccagcatgctagtgaatcatgtatcttttaggctgctgtgcatttctcttggcagtgatacctccctgacatggttcatcatcaggctgcaatgacagaatgtggtgagcagcgtctactgagactactaacattttgcactgtcaaaatacttggtgaggaaaagatagctcaggttattgctaatgggttaatgcaccagcaagcaaaatattttatgttttgggggttttgaaaaatcaaagataattaaccaaggatcttaactgtgttcgcattttttatccaagcacttagaaaacctacaatcctaattttgatgtccattgttaagaggtggtgatagatactattttttttttcatattgtatagcggttattagaaaagttggggattttcttgatctttattgctgcttaccattgaaacttaacccagctgtgttccccaactctgttctgcgcacgaaacagtatctgtttgaggcataatcttaagtggccacacacaatgttttctcttatgttatctggcagtaactgtaacttgaattacattagcacattctgcttagctaaaattgttaaaataaactttaataaacccatgtagccctctcatttgattgacagtattttagttatttttggcattcttaaagctgggcaatgtaatgatcagatctttgtttgtctgaacaggtatttttatacatgctttttgtaaaccaaaaacttttaaatttcttcaggttttctaacatgcttaccactgggctactgtaaatgagaaaagaataaaattatttaatgttttaa

SEQ ID NO: 5 - DNA, 3378 bp, Homo sapiens
ggcggcgccagcggacaccggagcgggcaggagcagcggccgcggcgccgcagggaccagcgggcccaggctcggtctggggttcttgcagctggcaccagccctcccggccccggccgcccacccgccgcggggaagatgccggaacagagtaacgattaccgcgtggtggtgttcggggcgggcggcgtgggcaagagctcgctggtgctgcgcttcgtgaagggcacgttccgcgacacctacatccccaccatcgaggacacctaccggcaggtgatcagctgcgacaagagcgtgtgcacgctgcagatcacagacaccaccggcagccaccagttcccggccatgcagcgcctgtccatctccaagggccacgccttcatcctggtgttctccgtcaccagcaagcagtcgctggaggagctggggcccatctacaagctcatcgtgcagatcaagggcagcgtggaggacatccccgtgatgctcgtgggcaacaagtgcgatgagacgcagcgggaggtggacacgcgcgaggcgcaggcggtggcccaggagtggaagtgcgctttcatggagacctcggccaagatgaactacaacgtcaaggagctcttccaggagctgctgacgctggagacgcgccggaacatgagcctcaacatcgacggcaagcgctccgggaagcagaagaggacagaccgcgtcaagggcaaatgcaccctcatgtgagcccggaacgcccgcctgcccgccgcccacccccactgacccccgctgcctcccccaacaccgacaccctcctcggcctcctcctcttctctcatccttcctccgacacctcggctggggaaaccgaggccaccgcccccccctccgctgcccctgcccaccccgaggcagggctggggcttttcttcctcccctgctctctctccaccctcctgttctgtctgtaccctccaaaaccaagagccggaggtggcccccttgtcctgcagatgggaaaacaggatggggagctggcaagaggagctgcttgttcccaccaggaccagaggaggctgcgtttccccgtttccatctctttccctggggtgtccccagccagacctgcgcgtcctgtccctcacatttgatcactgtgaccttctgggggaggggggagttgaaaatgcacatcggcctcagatatttttttcttttttctcctatttggtgttaacatacacccaagcccacccggcccgtcgtgacctctgatctgtgcccactcctccggttccagacgcacctctctcctctgtcttcacagtggggtgtggggcccgtgggatgggcctcaggccaccaggcaataaccacagggcctgcagcagtgcccctgccagccccgaatcccacccccgggaccagccacatccacagcacaactgccccgctggagaggcaccatgggcgtggaggggcttcccggacaccgcccacccgggacccgcctcttccaccaagacagagacgttagcaacgcatggcgggtggggacctggggtgctcaggagggggtacccggggccccggccagagatacatcaattacacccccgtggggggacagccgatgggagccagcaccagcaggatccgagggcgccccggacagaggtctgccccacccacttcctccccaccacctgtgccccagagagcagggcctgcccgggaaggtggcgtcctggagtcgagtgtacctgcagccatgaggttctgggtgttttttgagagagtctgagtgacaccacactcgtgtgaccccacagggttgtgtccaacatacacggaagtggctatggaatggtgtatttgtgcaacctggggtgcgcggatgggtgacttgtatctaagtgcatctgcgtgtatacctgtgtgtgtctgtctgggatgatatgtttttgtggcagtctgtgtgtgtaatagtggtgtagggtatacagagaggtgggtagttgtagatacctgtgtgtggttgtcagcaagactggatatgtgtgaggtgtctgtgtgaatctttgtgcctgtatgagcatgactatattttggggagtgggtgatatggtttatctgagagcatttatctgtaaatatgtttgtcctgattgagggacacgatctgtgttccactctatagcaacatgactctagcaatgtgactttcggttccaaatctgtatcagtcagctactgctgtgtaacaaatgaccacaaatgtagcaaccagaaacaacacatgcttattatctcatagattctgtgggtcaagagcctgggtgcaggttggctgggtcctctacttgggatctcaggaggctgcaatcaaagcattcgccaggcagaggtctcatctgaaggcctgatcggggaaggatttgcttcttagaagctcatgtggttgttgcagcattcagttccttgctgttgcaagactgaaggcctcagttcctcgctggctgttggctggaagctgccctttgttctgtaccatgtgggtctctccacagcggggctcggagcatggcagctaagttagtgagggaaggtgagatggaggttttggtcttattgggtgtgaggaagcaacgtgtgtgtgtgcgcacgcccttttgtgcagtgagagagagagagagattgcacacatgtgtctctgtagtcatgtggccaggtgggactatgtaggtaacagattgctcgtgtctgatttggtacaagcatgtttgttttcctctgtgttcgtgtgagtgtttactcaacaaatgtttattggacacactcagagagagggagtgtgcacacgtgcgtgtgtgttgctatccagcacgtggaccgggctcccagaagagctggcattgtgtctgagcagagctgggtccccccaaaacttgggctggcccagggcccaccagcagctgatgttgcctcctctcctgtcctggcagtagcttctgggttctgaaggtgccggagagagtgaggctgggcaggggtctgcggccctttctcagggacacaccctgatagcacaatctccttggggccctgcccacctccaggcctctcccacctcaggccctgcccgaccctggggagagagggcatctgcaataggaggggacccgagcctgtcctggctgctggcccatcctgcctgggcatccctggtgctggggactgtgccaggccatgcttgctgtgactccgcccctgccccctctccccccgcatgtgggtgcccccactcccccatcgtggggtctgtgtagccttcgctctagacatagtcttcctgcaataaaaaagtggatcctgcattccccacca

SEQ ID NO: 6 - DNA, 2809 bp, Homo sapiens
ctgcggaatgtcaactattcaacatggaggcggaggtcgataagctggaactgatgttccagaaagctgagtctgatctggattacattcaatacaggctggaatatgaaatcaagactaatcatcctgattcagcaagtgagaaaaatccagttacactcttaaaggaattgtcagtgataaagtctcgatatcaaactttgtatgcccgctttaaaccagttgctgttgagcagaaagagagtaagagccgcatttgtgctactgtgaaaaagactatgaatatgatacaaaaactacagaagcaaacagacctggagctgtcaccactgactaaagaagagaaaactgcggcagagcaattcaaatttcacatgccagatttatgaagaaatggacttggaaaggaaattctaacagagaagagcttaattccggagaaatttaggaagatgtcttgttaacccttgatgtctagagattgggggctggtgaagggggtttggcttcaatgactggataatgatatctttcatgagagagattataagaagaagggcagataatatatgaataaagttcagccaaaaggatcaaatgagaataaaacgatttaaatatatgtacacacgcatgcacacacacacttagtcttgtaatttcaggccagaaattctcaacactattttgcatctgttttctttttctaagtcatgataatatagatgttctggtctatcataaaagaatgtttatgtacatttcagtcattcggtatgtggctttgtaaattaaagtataggcaaaacatttgtgttatacatgatatataatttcattttgtaaatgttgattgcacatgtggtcacattattgttgagactgcttttatgtgacctgtagtctcccacagaacctaaagtaataagctggcttttctgtgatagccacgtttgcgtatttctttccctatttcccttgcctgctaatgttgaacagcatgaacttgctttctgatgctgttttagactgtccctgttgtatctcaataatatctttgttttccttcagcctttattactataattgttcattctacatgaaagctaggaaactgaaattagaagagcacttatctgctacttgccagttttgcgtgagtgtgttatatgtatgtgtcaatttccctttaaaataactatttattttaaaataactattggcaataaggaaactgttcaaagtagaggcagatcttgatagaaagatgttaatcacagggttgtttataatagcaatatacatacacatttggctagtactaggtgaataggaaaataaatcatgctgtatgtatacaataagaggtcaagttgccaataaattattactgttaatgttctgggaaatgctgaaactatgctaagtgggggagagggaaagcaggtattgcagttttgtagtgaagattgggctttggagtcatatctgagatgtaagtagcagcttttaaattctagctatgaccctgtgcagatcacttaacttttgagtgtcaggatgtttggaaggacaagacaggaaagtgttttaatacagggttcagtatttagtaagccttcaataagtgatattacatacatgtaataaaaactaagcattgaaaggactaggaaaaatgcatcaaatgttagttctgcaggttatagaattatacttttatgtaattttaaaatttcagttatgaacatgtttgtccattaacaaacttataatagctgaaaaacagggttgtgagaacagcttcagttaaaattgtcctgtctagtttgaatatttcaagaacagaaagcctttttttttttttttttttttttgagacggagtctcactctgttgcccaggctggaatgcagtggtacgctctcagctcactgcaacctccacctcccaggttcaagcagttctctgccccaccctcccgagtagctgggattacaggcgcccgccaccacgcccggctatttttttgtatttttagtagagatggggcttcaccatcttgaccaggctggtctttaactcctgatggtgatccacccacctcggcctcccaaagtgctgggattacaggcatgagctaccgtgcctggcccccttttttttaattacagagaaataagttacaccttagtatcagatattaattttcttcagtgttcaggcaattagtatttagaaagctcttgtcatgagatggctctgggatgtgatgatgattgttgggattgaaaaaacggtagtatcatggagagatcataataaattcttagtattaaaagtggttttgctttcagttagggagaaaaattagattgtactatttttcctctatgatttccttcagttatcttccaaatgttgttttttccccacagcccccttaacattgttctctatgcacttctcaatacattttcatttgtttctcaagcctctttgtggatgactcctaaatataacttcttccactagctctagatccgtatttccaataaaatcccctacctgaatattcaagttaaacatgtccagaatacttactgattttattgttaatagccactattctgttgtctggaattaaaacctgtataactaatttgcatccctttatcttcttagtcaataaaacctacaatcctctttctaaaatggtcatagtatacaacctctacattatcttcacacctccttaatttagatcttttgtttctcatttggattataattgttttctcctatgtga

SEQ ID NO: 7 - DNA, 2829 bp, Homo sapiens
ctgcggaatgtcaactattcaacatggaggcggaggtcgataagctggaactgatgttccagaaagctgagtctgatctggattacattcaatacaggctggaatatgaaatcaagactaatcatcctgattcagcaagtgagaaaaatccagttacactcttaaaggaattgtcagtgataaagtctcgatatcaaactttgtatgcccgctttaaaccagttgctgttgagcagaaagagagtaagagccgcatttgtgctactgtgaaaaagactatgaatatgatacaaaaactacagaagcaaacagacctggaggtaatgctttcagttgacagctgtcaccactgactaaagaagagaaaactgcggcagagcaattcaaatttcacatgccagatttatgaagaaatggacttggaaaggaaattctaacagagaagagcttaattccggagaaatttaggaagatgtcttgttaacccttgatgtctagagattgggggctggtgaagggggtttggcttcaatgactggataatgatatctttcatgagagagattataagaagaagggcagataatatatgaataaagttcagccaaaaggatcaaatgagaataaaacgatttaaatatatgtacacacgcatgcacacacacacttagtcttgtaatttcaggccagaaattctcaacactattttgcatctgttttctttttctaagtcatgataatatagatgttctggtctatcataaaagaatgtttatgtacatttcagtcattcggtatgtggctttgtaaattaaagtataggcaaaacatttgtgttatacatgatatataatttcattttgtaaatgttgattgcacatgtggtcacattattgttgagactgcttttatgtgacctgtagtctcccacagaacctaaagtaataagctggcttttctgtgatagccacgtttgcgtatttctttccctatttcccttgcctgctaatgttgaacagcatgaacttgctttctgatgctgttttagactgtccctgttgtatctcaataatatctttgttttccttcagcctttattactataattgttcattctacatgaaagctaggaaactgaaattagaagagcacttatctgctacttgccagttttgcgtgagtgtgttatatgtatgtgtcaatttccctttaaaataactatttattttaaaataactattggcaataaggaaactgttcaaagtagaggcagatcttgatagaaagatgttaatcacagggttgtttataatagcaatatacatacacatttggctagtactaggtgaataggaaaataaatcatgctgtatgtatacaataagaggtcaagttgccaataaattattactgttaatgttctgggaaatgctgaaactatgctaagtgggggagagggaaagcaggtattgcagttttgtagtgaagattgggctttggagtcatatctgagatgtaagtagcagcttttaaattctagctatgaccctgtgcagatcacttaacttttgagtgtcaggatgtttggaaggacaagacaggaaagtgttttaatacagggttcagtatttagtaagccttcaataagtgatattacatacatgtaataaaaactaagcattgaaaggactaggaaaaatgcatcaaatgttagttctgcaggttatagaattatacttttatgtaattttaaaatttcagttatgaacatgtttgtccattaacaaacttataatagctgaaaaacagggttgtgagaacagcttcagttaaaattgtcctgtctagtttgaatatttcaagaacagaaagcctttttttttttttttttttttttgagacggagtctcactctgttgcccaggctggaatgcagtggtacgctctcagctcactgcaacctccacctcccaggttcaagcagttctctgccccaccctcccgagtagctgggattacaggcgcccgccaccacgcccggctatttttttgtatttttagtagagatggggcttcaccatcttgaccaggctggtctttaactcctgatggtgatccacccacctcggcctcccaaagtgctgggattacaggcatgagctaccgtgcctggcccccttttttttaattacagagaaataagttacaccttagtatcagatattaattttcttcagtgttcaggcaattagtatttagaaagctcttgtcatgagatggctctgggatgtgatgatgattgttgggattgaaaaaacggtagtatcatggagagatcataataaattcttagtattaaaagtggttttgctttcagttagggagaaaaattagattgtactatttttcctctatgatttccttcagttatcttccaaatgttgttttttccccacagcccccttaacattgttctctatgcacttctcaatacattttcatttgtttctcaagcctctttgtggatgactcctaaatataacttcttccactagctctagatccgtatttccaataaaatcccctacctgaatattcaagttaaacatgtccagaatacttactgattttattgttaatagccactattctgttgtctggaattaaaacctgtataactaatttgcatccctttatcttcttagtcaataaaacctacaatcctctttctaaaatggtcatagtatacaacctctacattatcttcacacctccttaatttagatcttttgtttctcatttggattataattgttttctcctatgtga

SEQ ID NO: 8 - DNA, 2807 bp, Homo sapiens
ctgcggaatgtcaactattcaacatggaggcggaggtcgataagctggaactgatggtgagtgcaaagtgaaggagatctactgggtagtttatttttcccccatggcctcggaggtggggcacaatttggagtcgccggaaactccgggcggcggaggctggaccagagtcgagttccctcctcctgcaccaaagggagccgccaccgtctggtgtctaaaccgcctcggttccagaaagctgagtctgatctggattacattcaatacaggctggaatatgaaatcaagactaatcatcctgattcagcaagtgagctgtcaccactgactaaagaagagaaaactgcggcagagcaattcaaatttcacatgccagatttatgaagaaatggacttggaaaggaaattctaacagagaagagcttaattccggagaaatttaggaagatgtcttgttaacccttgatgtctagagattgggggctggtgaagggggtttggcttcaatgactggataatgatatctttcatgagagagattataagaagaagggcagataatatatgaataaagttcagccaaaaggatcaaatgagaataaaacgatttaaatatatgtacacacgcatgcacacacacacttagtcttgtaatttcaggccagaaattctcaacactattttgcatctgttttctttttctaagtcatgataatatagatgttctggtctatcataaaagaatgtttatgtacatttcagtcattcggtatgtggctttgtaaattaaagtataggcaaaacatttgtgttatacatgatatataatttcattttgtaaatgttgattgcacatgtggtcacattattgttgagactgcttttatgtgacctgtagtctcccacagaacctaaagtaataagctggcttttctgtgatagccacgtttgcgtatttctttccctatttcccttgcctgctaatgttgaacagcatgaacttgctttctgatgctgttttagactgtccctgttgtatctcaataatatctttgttttccttcagcctttattactataattgttcattctacatgaaagctaggaaactgaaattagaagagcacttatctgctacttgccagttttgcgtgagtgtgttatatgtatgtgtcaatttccctttaaaataactatttattttaaaataactattggcaataaggaaactgttcaaagtagaggcagatcttgatagaaagatgttaatcacagggttgtttataatagcaatatacatacacatttggctagtactaggtgaataggaaaataaatcatgctgtatgtatacaataagaggtcaagttgccaataaattattactgttaatgttctgggaaatgctgaaactatgctaagtgggggagagggaaagcaggtattgcagttttgtagtgaagattgggctttggagtcatatctgagatgtaagtagcagcttttaaattctagctatgaccctgtgcagatcacttaacttttgagtgtcaggatgtttggaaggacaagacaggaaagtgttttaatacagggttcagtatttagtaagccttcaataagtgatattacatacatgtaataaaaactaagcattgaaaggactaggaaaaatgcatcaaatgttagttctgcaggttatagaattatacttttatgtaattttaaaatttcagttatgaacatgtttgtccattaacaaacttataatagctgaaaaacagggttgtgagaacagcttcagttaaaattgtcctgtctagtttgaatatttcaagaacagaaagcctttttttttttttttttttttttgagacggagtctcactctgttgcccaggctggaatgcagtggtacgctctcagctcactgcaacctccacctcccaggttcaagcagttctctgccccaccctcccgagtagctgggattacaggcgcccgccaccacgcccggctatttttttgtatttttagtagagatggggcttcaccatcttgaccaggctggtctttaactcctgatggtgatccacccacctcggcctcccaaagtgctgggattacaggcatgagctaccgtgcctggcccccttttttttaattacagagaaataagttacaccttagtatcagatattaattttcttcagtgttcaggcaattagtatttagaaagctcttgtcatgagatggctctgggatgtgatgatgattgttgggattgaaaaaacggtagtatcatggagagatcataataaattcttagtattaaaagtggttttgctttcagttagggagaaaaattagattgtactatttttcctctatgatttccttcagttatcttccaaatgttgttttttccccacagcccccttaacattgttctctatgcacttctcaatacattttcatttgtttctcaagcctctttgtggatgactcctaaatataacttcttccactagctctagatccgtatttccaataaaatcccctacctgaatattcaagttaaacatgtccagaatacttactgattttattgttaatagccactattctgttgtctggaattaaaacctgtataactaatttgcatccctttatcttcttagtcaataaaacctacaatcctctttctaaaatggtcatagtatacaacctctacattatcttcacacctccttaatttagatcttttgtttctcatttggattataattgttttctcctatgtga

SEQ ID NO: 9 - DNA, 1547 bp, Homo sapiens
gaagaaacatccaaggtggtcttgaaggacactgggatcctgtaacacagccccggatatctgtgttaccagccttgtctcggccacctcaaggataatcactaaattctgccgaaaggactgaggaacggtgcctggaaaagggcaagaatatcacggcatgggcatgagtagcttgaaactgctgaagtatgtcctgtttttcttcaacttgctcttttggatctgtggctgctgcattttgggctttgggatctacctgctgatccacaacaacttcggagtgctcttccataacctcccctccctcacgctgggcaatgtgtttgtcatcgtgggctctattatcatggtagttgccttcctgggctgcatgggctctatcaaggaaaacaagtgtctgcttatgtcgttcttcatcctgctgctgattatcctccttgctgaggtgaccttggccatcctgctctttgtatatgaacagaagctgaatgagtatgtggctaagggtctgaccgacagcatccaccgttaccactcagacaatagcaccaaggcagcgtgggactccatccagtcatttctgcagtgttgtggtataaatggcacgagtgattggaccagtggcccaccagcatcttgcccctcagatcgaaaagtggagggttgctatgcgaaagcaagactgtggtttcattccaatttcctgtatatcggaatcatcaccatctgtgtatgtgtgattgaggtgttggggatgtcctttgcactgaccctgaactgccagattgacaaaaccagccagaccatagggctatgatctgcagtagtcctgtggtgaagagacttgtttcatctccggaaatgcaaaaccatttatagcatgaagccctacatgatcactgcaggatgatcctcctcccatcctttccctttttaggtccctgtcttatacaaccagagaagtgggtgttggccaggcacatcccatctcaggcagcaagacaatctttcactcactgacggcagcagccatgtctctcaaagtggtgaaactaatatctgagcatcttttagacaagagaggcaaagacaaactggatttaatggcccaacatcaaagggtgaacccaggatatgaatttttgcatcttcccattgtcgaattagtctccagcctctaaataatgcccagtcttctccccaaagtcaagcaagagactagttgaagggagttctggggccaggctcactggaccattgtcacaaccctctgtttctctttgactaagtgccctggctacaggaattacacagttctctttctccaaagggcaagatctcatttcaatttctttattagagggccttattgatgtgttctaagtctttccagaaaaaaactatccagtgatttatatcctgatttcaaccagtcacttagctgataatcacagtaagaagacttctggtattatctctctatcagataagattttgttaatgtactattttactcttcaataaataaaagtttattatctcaa

SEQ ID NO: 10 - DNA, 1505 bp, Homo sapiens
acaaatagccccggatatctgtgttaccagccttgtctcggccacctcaaggataatcactaaattctgccgaaaggactgaggaacggtgcctggaaaagggcaagaatatcacggcatgggcatgagtagcttgaaactgctgaagtatgtcctgtttttcttcaacttgctcttttggatctgtggctgctgcattttgggctttgggatctacctgctgatccacaacaacttcggagtgctcttccataacctcccctccctcacgctgggcaatgtgtttgtcatcgtgggctctattatcatggtagttgccttcctgggctgcatgggctctatcaaggaaaacaagtgtctgcttatgtcgttcttcatcctgctgctgattatcctccttgctgaggtgaccttggccatcctgctctttgtatatgaacagaagctgaatgagtatgtggctaagggtctgaccgacagcatccaccgttaccactcagacaatagcaccaaggcagcgtgggactccatccagtcatttctgcagtgttgtggtataaatggcacgagtgattggaccagtggcccaccagcatcttgcccctcagatcgaaaagtggagggttgctatgcgaaagcaagactgtggtttcattccaatttcctgtatatcggaatcatcaccatctgtgtatgtgtgattgaggtgttggggatgtcctttgcactgaccctgaactgccagattgacaaaaccagccagaccatagggctatgatctgcagtagtcctgtggtgaagagacttgtttcatctccggaaatgcaaaaccatttatagcatgaagccctacatgatcactgcaggatgatcctcctcccatcctttccctttttaggtccctgtcttatacaaccagagaagtgggtgttggccaggcacatcccatctcaggcagcaagacaatctttcactcactgacggcagcagccatgtctctcaaagtggtgaaactaatatctgagcatcttttagacaagagaggcaaagacaaactggatttaatggcccaacatcaaagggtgaacccaggatatgaatttttgcatcttcccattgtcgaattagtctccagcctctaaataatgcccagtcttctccccaaagtcaagcaagagactagttgaagggagttctggggccaggctcactggaccattgtcacaaccctctgtttctctttgactaagtgccctggctacaggaattacacagttctctttctccaaagggcaagatctcatttcaatttctttattagagggccttattgatgtgttctaagtctttccagaaaaaaactatccagtgatttatatcctgatttcaaccagtcacttagctgataatcacagtaagaagacttctggtattatctctctatcagataagattttgttaatgtactattttactcttcaataaataaaagtttattatctcaa

SEQ ID NO: 11 - DNA, 1328 bp, Homo sapiens
acaaatagccccggatatctgtgttaccagccttgtctcggccacctcaaggataatcactaaattctgccgaaaggactgaggaacggtgcctggaaaagggcaagaatatcacggcatgggcatgagtagcttgaaactgctgaagtatgtcctgtttttcttcaacttgctcttttggatctgtggctgctgcattttgggctttgggatctacctgctgatccacaacaacttcggagtgctcttccataacctcccctccctcacgctgggcaatgtgtttgtcatcgtgggctctattatcatggtagttgccttcctgggctgcatgggctctatcaaggaaaacaagtgtctgcttatgtcgttcttcatcctgctgctgattatcctccttgctgaggtgaccttggccatcctgctctttgtatatgaacagaagggttgctatgcgaaagcaagactgtggtttcattccaatttcctgtatatcggaatcatcaccatctgtgtatgtgtgattgaggtgttggggatgtcctttgcactgaccctgaactgccagattgacaaaaccagccagaccatagggctatgatctgcagtagtcctgtggtgaagagacttgtttcatctccggaaatgcaaaaccatttatagcatgaagccctacatgatcactgcaggatgatcctcctcccatcctttccctttttaggtccctgtcttatacaaccagagaagtgggtgttggccaggcacatcccatctcaggcagcaagacaatctttcactcactgacggcagcagccatgtctctcaaagtggtgaaactaatatctgagcatcttttagacaagagaggcaaagacaaactggatttaatggcccaacatcaaagggtgaacccaggatatgaatttttgcatcttcccattgtcgaattagtctccagcctctaaataatgcccagtcttctccccaaagtcaagcaagagactagttgaagggagttctggggccaggctcactggaccattgtcacaaccctctgtttctctttgactaagtgccctggctacaggaattacacagttctctttctccaaagggcaagatctcatttcaatttctttattagagggccttattgatgtgttctaagtctttccagaaaaaaactatccagtgatttatatcctgatttcaaccagtcacttagctgataatcacagtaagaagacttctggtattatctctctatcagataagattttgttaatgtactattttactcttcaataaataaaagtttattatctcaa

SEQ ID NO: 12 - DNA, 962 bp, Homo sapiens
gtagtggcgccgggagtcgcgggtgcgcgcgggccgtgagtgtgcgcttttgagagtcgcggcggaaggagcccggccgccgcccgccggcatgagctacgaccgcgccatcaccgtcttctcgcccgacggccacctcttccaagtggagtacgcgcaggaggccgtcaagaagggctcgaccgcggttggtgttcgaggaagagacattgttgttcttggtgtggagaagaagtcagtggccaaactgcaggatgaaagaacagtgcggaagatctgtgctttggatgacaacgtctgcatggcctttgcaggcctcaccgccgatgcaaggatagtcatcaacagggcccgggtggagtgccagagccaccggctgactgtggaggacccggtcactgtggagtacatcacccgctacatcgccagtctgaagcagcgttatacgcagagcaatgggcgcaggccgtttggcatctctgccctcatcgtgggtttcgactttgatggcactcctaggctctatcagactgacccctcgggcacataccatgcctggaaggccaatgccataggtcggggtgccaagtcagtgcgcgagttcctggagaagaactatactgacgaagccattgaaacagatgatctgaccattaagctggtgatcaaggcactcctggaagtggttcagtcaggtggcaaaaacattgaacttgctgtcatgaggcgagatcaatccctcaagattttaaatcctgaagaaattgagaagtatgttgctgaaattgaaaaagaaaaagaagaaaacgaaaagaagaaacaaaagaaagcatcatgatgaataaaatgtctttgcttgtaatttttaaattcatatcaatcatggatgagtctcgatgtgtaggcctttccattccatttattcacactgagtgtcctacaataaacttccgtatttttaa

SEQ ID NO: 13 - DNA, 1567 bp, Homo sapiens
aagtgcttcgcggcggaggcccgggcaactcttttgaatggaatcgggctgattcatcgccggtttgcagacagagccgcgtcgggtgtgcgccgctgctgctgttgcctctgtcttcgcgtcaccacagaggcaagacaagggtccatatcgcggcatccggctcccgcccgtcttcaggagagaaagaaaaaataaaatatacttggggaagttgtacctgccagaattagcaagagctttctttaagaagacatttgtcaaactcaacaaattgaaggttaacaccttaagagttgtagttactgaccagaaatatggacagacttcttagacttggaggaggtatgcctggactgggccaggggccacctacagatgctcctgcagtggacacagcagaacaagtctatatctcttccctggcactgttaaaaatgttaaaacatggccgtgctggagttccaatggaagttatgggtttgatgcttggagaatttgttgatgattataccgtcagagtgattgatgtgtttgctatgccacagtcaggaacaggtgtcagtgtggaggcagttgatccagtgttccaagctaaaatgttggatatgttgaagcagacaggaaggccggagatggttgttggttggtatcacagtcaccctggctttggttgttggctttctggtgtggatatcaacactcagcagagctttgaagccttgtcggagagagctgtggcagtggttgtggatcccattcagagtgtaaaaggaaaggttgttattgatgccttcagattgatcaatgctaatatgatggtcttaggacatgaaccaagacaaacaacttcgaatctgggtcacttaaacaagccatctatccaggcattaattcatggactaaacagacattattactccattactattaactatcggaaaaatgaactggaacagaagatgttgctaaatttgcataagaagagttggatggaaggtttgacacttcaggactacagtgaacattgtaaacacaatgaatcagtggtaaaagagatgttggaattagccaagaattacaataaggctgtagaagaagaagataagatgacacctgaacagctggcaataaagaatgttggcaagcaggaccccaaacgtcatttggaggaacatgtggatgtacttatgacctcaaatattgtccagtgtttagcagctatgttggatactgtcgtatttaaataaagcaacgaaaaacgctattaatgatgccttcagtgtatattcctctgttgttcctaatgctcaaaatcaagggacctctgaaggtgtacttggctaaatgtaagacatctggcatcatttgcagcactgtaacaccttcagtctcagttgtgcaattacttctgtttctttagtcagggtctttgcagattctaaagttatacatgaatacatcaaagtggacaaattttgttaagatcccatttaatatttgaaaaaatcagtagcacaaatatattttgattgtcacttacaaaataaaatacatttacagtcta

(19) United States
(12) Patent Application Publication
(10) Pub. No.: US 2024/0150843 A1
(43) Pub. Date: May 9, 2024

(54) METHODS AND MATERIALS FOR IDENTIFYING MYELOMA STAGE AND DRUG SENSITIVITY AND TREATING MYELOMA

(71) Applicant: Mayo Foundation for Medical Education and Research, Rochester, MN (US)

(72) Inventors: Xianfeng Chen, Scottsdale, AZ (US); Yuan Xiao Zhu, Scottsdale, AZ (US); Alexander Keith Stewart, Fountain Hills, AZ (US); Lisa M. Rimsza, Scottsdale, AZ (US)

(21) Appl. No.: 18/282,467

(22) PCT Filed: Mar. 17, 2022
(86) PCT No.: PCT/US2022/020767

§ 371 Date: Sep. 15, 2023

Related U.S. Application Data
(60) Provisional application No. 63/162,314, filed on Mar. 17, 2021.

(51) Int. Cl. C12Q 1/6886

(52) U.S. Cl. CPC C12Q 1/6886; C12Q 2600/158

(57) ABSTRACT

Methods and materials for identifying and treating mammals (e.g., humans) having multiple myeloma (MM) are provided herein. For example, this document provides methods and materials that can be used to identify and treat mammals having advanced stage MM, and/or having MVI that is resistant to treatment with immunomodulatory drugs (IMiDs) and/or proteasome inhibitors (PIs).

### CROSS-REFERENCE TO RELATED APPLICATIONS

This application claims benefit of priority from U.S. Provisional Application No. 63/162,314, filed on Mar. 17, 2021. The disclosure of the prior application is considered part of (and is incorporated by reference in) the disclosure of this application.

### STATEMENT AS TO FEDERALLY SPONSORED RESEARCH

This invention was made with government support under CA224018 awarded by the National Institutes of Health. The government has certain rights in the invention.

### SEQUENCE LISTING

This application contains a Sequence Listing that has been submitted electronically as an ASCII text file named SequenceListing.txt. The ASCII text file, created on Mar. 15, 2022, is 38.2 kilobytes in size. The material in the ASCII text file is hereby incorporated by reference in its entirety.

### TECHNICAL FIELD

This document relates to methods and materials for identifying and treating mammals (e.g., humans) having multiple myeloma (MM). For example, this document relates to methods and materials for identifying and treating mammals having advanced stage MM, and/or having MM that is resistant to treatment with immunomodulatory drugs (IMiDs) and/or proteasome inhibitors (PIs).

### BACKGROUND

The introduction of IMiDs and PIs changed the therapeutic paradigm for treatment of MM. IMiDs can mediate anti-MM effects by binding to the E3 ubiquitin ligase, cereblon (CRBN) (Ito et al., Science 327(5971):1345-1350, 2010; Zhu et al., Blood 118(18):4771-4779, 2011; and Lopez-Girona et al., Leukemia 26(11):2326-2335, 2012), which subsequently increases degradation of the transcription factors Ikaros (IKZF1) and Aiolos (IKZF3), culminating in downregulation of IRF4 and MYC expression and inhibition of MM cell growth (Kronke et al., Science 343(6168):301-305, 2014; and Lu et al., Science 343(6168):305-309, 2014).

While the majority of patients with MM initially respond to IMiDs and PIs, most eventually develop resistance. Resistance to IMiDs in MM has been linked to the deletion, functional mutation, or dysregulation of CRBN and the proteins directly and indirectly associated with CRBN or IMiD-mediated signaling (Zhu et al., supra; Kortum et al., Blood 128(9):1226-1233, 2016; Zhu et al., Blood Cancer J 9(2):19, 2019; Ocio et al., Leukemia 29(3):705-714, 2015; Dimopoulos et al., Mol Oncol 12(2):180-195, 2018; Franssen et al., Haematologica 103(8):e368-e371, 2018; Schuster et al., Leuk Res 38(1):23-28, 2014; and Zhou et al., Leukemia 33(8):2006-2021, 2019). Resistance to PIs in MM is thought to be due, at least in part, to mutation and/or dysregulation of proteasome subunits (Kale and Moore, J Med Chem 55(23):10317-10327, 2012; Ling et al., Haematologica 97(1):64-72, 2012; Nikesitch and Ling, J Clin Pathol 69(2):97-101, 2016; Shi et al., Mol Cancer Ther 16(12):2862-2870, 2017; Barrio et al., Leukemia 33(2):447-456, 2019; Soriano et al., Leukemia 30(11):2198-2207, 2016; and Besse et al., Leukemia 32(2):391-401, 2018).

### SUMMARY

This document provides methods and materials for identifying and treating mammals (e.g., humans) having MM. For example, this document provides methods and materials for identifying and treating mammals having advanced stage MM, and/or having MM that is resistant to treatment with IMiDs and/or PIs.

In general, this document features methods for treating a mammal having MM. The methods can include, or consist essentially of: (a) identifying a mammal as having a biological sample with an altered level of expression of one or more markers as compared to a level of expression of the one or more markers in a biological sample from a corresponding mammal that does not have late stage MM or refractory/resistant (RR) MM, wherein the one or more markers are selected from the group consisting of CRBN, CEP55, DIRAS1, SKA2, CD53, PSMA7 and PSMD14, thereby identifying the mammal as having or being likely to have late stage MM or RR MM, and (b) administering to the mammal a composition comprising chimeric antigen receptor- (CAR-) T cells or a histone deacetylase (HDAC) inhibitor. The mammal can be a human. The biological sample can be a blood sample obtained from the mammal. The blood sample can be a plasma sample. The methods can include identifying the biological sample as having altered levels of expression of two or more of the markers, as compared to levels of expression of the two or more markers in the biological sample from the corresponding mammal. The methods can include identifying the biological sample as having reduced expression of one or more markers selected from the group consisting of CRBN, DIRAS1, CD53, and SKA2, as compared to expression in the biological sample from the corresponding mammal. The methods can include identifying the biological sample as having elevated expression of one or more markers selected from the group consisting of CEP55, PSMA7, and PSMD14, as compared to expression in the biological sample from the corresponding mammal. The methods can include identifying the biological sample as having reduced expression of CRBN, DIRAS1, CD53, and SKA2, and elevated expression of CEP55 and PSMD14, as compared to expression of the markers in the biological sample from the corresponding mammal. The identifying can include comprises using NanoString nCounter technology to detect expression of the one or more markers in the biological sample.

In another aspect, this document features methods for treating a mammal having MM, where the mammal was identified as having a biological sample with an altered level of expression of one or more markers as compared to a level of expression of the one or more markers in a biological sample from a corresponding mammal that does not have late stage MM or RR MM, and where the one or more markers are selected from the group consisting of CRBN, CEP55, DIRAS1, SKA2, CD53, PSMA7 and PSMD14. The methods can include administering to the mammal a composition comprising CAR-T cells or a HDAC inhibitor, wherein one or more symptoms of the MM is reduced in the mammal. The mammal can be a human. The biological sample can be a blood sample obtained from the mammal. The blood sample can be a plasma sample. The mammal can have been identified as having a biological sample with altered levels of expression of two or more of the markers, as compared to levels of expression of the two or more markers in the biological sample from the corresponding mammal. The mammal can have been identified as having a biological sample with reduced expression of one or more markers selected from the group consisting of CRBN, DIRAS1, CD53, and SKA2, as compared to expression in the biological sample from the corresponding mammal. The mammal can have been identified as having a biological sample with elevated expression of one or more markers selected from the group consisting of CEP55, PSMA7, and PSMD14, as compared to expression in the biological sample from the corresponding mammal. The mammal can have been identified as having a biological sample as with reduced expression of CRBN, DIRAS1, CD53, and SKA2, and elevated expression of CEP55 and PSMD14, as compared to expression of the markers in the biological sample from the corresponding mammal. The mammal can have been identified by using NanoString nCounter technology to detect expression of the one or more markers in the biological sample.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a schematic illustrating the primary MM samples and human MM cell lines HMCLs that were used for NanoString profiling. Patient samples were selected and grouped based on the stage of disease activity when samples were collected. Numbers in brackets indicate the number of probes for each gene or the number of patients in each group.

FIGS. 2A-2E show that NanoString technology provided a sensitive, reliable and reproducible method to quantitate gene expression changes in myeloma cells. FIG. 2A is a graph plotting the correlation of two biological repeats generated from the NanoString profiling of MM1.S cell lines. FIGS. 2B and 2C are graphs plotting levels of CRBN mRNA expression (FIG. 2B) and levels of IL6 mRNA expression (FIG. 2C) in two different lenalidomide isogenic resistant cell lines, as detected by NanoString profiling. FIG. 2D is a heatmap view of the normalized data from 4 pairs of isogenic IMiD sensitive/resistant cell lines. FIG. 2E is a graph plotting lenalidomide-mediated transcriptional responses of the indicated genes in the OCIMY5/CRBN (lenalidomide sensitive) cell line.

FIG. 3 includes a volcano plot showing differentially expressed genes for resistant cell lines vs. baseline of sensitive cell lines (4 pairs), along with a table listing the top 15 differentially expressed genes. The volcano plot displays each gene's −log₁₀ (p-value) and log₂ fold change with the selected covariate. Highly statistically significant genes fall at the top of the plot, and highly differentially expressed genes fall to either side. The most statistically significant genes are labeled in the plot.

FIGS. 4A and 4B show differentially expressed genes between newly diagnosed (ND) and late stage relapsed refractory (RR) samples. Each panel includes a volcano plot displaying each gene's −log₁₀ (p-value) and log₂ fold change with the selected covariate. Highly statistically significant genes fall at the top of the plot above the horizontal lines, and highly differentially expressed genes fall to either side. Horizontal lines indicate various p-value thresholds. The 20 most statistically significant genes are labeled in each plot, and the top 16 differentially expressed genes are shown in the table to the right of each plot. For FIG. 4A, 69 late/RR samples were compared with 52 ND samples, while for FIG. 4B, 11 paired late/RR samples and ND samples were compared.

FIGS. 5A and 5B show differentially expressed genes between newly diagnosed MM and samples harvested during active treatment. Each panel includes a volcano plot displaying each gene's −log₁₀ (p-value) and log₂ fold change with the selected covariate. Highly statistically significant genes fall at the top of the plot above the horizontal lines, and highly differentially expressed genes fall to either side. Horizontal lines indicate various p-value thresholds. The 20 most statistically significant genes are labeled in the plot, and the top 16 differentially expressed genes are shown in the table to the right of each plot. For FIG. 5A, 8 paired samples harvested at time of diagnosis and during or after treatment with IMiD-based therapy (no PIs were used) were compared. For FIG. 5B, 14 paired samples harvested at time of diagnosis and during or after treatment with IMiDs and PIs were compared.

FIG. 6 includes a volcano plot showing differentially expressed genes for paired PIs vs. ND samples (3 pairs), along with a table listing the top 15 differentially expressed genes. The volcano plot displays each gene's −log₁₀ (p-value) and log₂ fold change with the selected covariate. Highly statistically significant genes fall at the top of the plot, and highly differentially expressed genes fall to either side. The most statistically significant genes are labeled in the plot.

FIG. 7 includes a volcano plot showing differentially expressed genes for paired late samples vs. early samples (5 pairs), along with a table listing the top 15 differentially expressed genes. The volcano plot displays each gene's −log₁₀ (p-value) and log₂ fold change with the selected covariate. Highly statistically significant genes fall at the top of the plot, and highly differentially expressed genes fall to either side. The most statistically significant genes are labeled in the plot.

FIGS. 8A and 8B show differentially expressed genes between IMiD or PI sensitive and resistant HMCLs. Each panel includes a volcano plot displaying each gene's −log₁₀ (p-value) and log₂ fold change with the selected covariate. Highly statistically significant genes fall at the top of the plot above the horizontal lines, and highly differentially expressed genes fall to either side. Horizontal lines indicate various p-value thresholds. The 20 most statistically significant genes are labeled in the plot, and the top 16 differentially expressed genes are shown in the table to the right of each plot. For FIG. 8A, 6 lenalidomide resistant HMCLs were compared with 8 lenalidomide sensitive HMCLs. For FIG. 8B, 5 paired isogenic bortezomib sensitive and resistant HMCLs were compared.

FIGS. 9A-91I show correlation of the expression of selected genes with survival and drug response in the Multiple Myeloma Research Foundation (MMRF) CoMMpass datasets. The plots were prepared based on the RNAseq and clinical data in MMRF CoMMpass database (Explore 1A13). Each panel includes Kaplan-Meier curves for selected clinical endpoints (overall survival, progression free survival, and response duration) with censoring, showing the estimated probability over time for all patients in the dataset. The plots show the differential probabilities between the patients belonging to the selected groups (colored and grouped based on RPKM value, numbers in brackets indicate the number of patients in each group). A test of equal hazards between groups was performed and the p-value for the log-rank is displayed along with the hazard ratio between each pairwise group. FIG. 9A, PRR11; FIG. 9B, PKB; FIG. 9C, DEPCD1; FIG. 9D, BIRC5; FIG. 9E, LTBP1, FIG. 9F, ITPRIPL2; FIG. 9G, TNFRSF13C and FIG. 9H, RNFT2.

FIG. 10 is a pair of graphs demonstrating the correlation of PRR11 expression (based on RPKM value) with drug treatment (left) and survival (right) in the Mayo Clinic myeloma data set. The plots were prepared based on the RNAseq and clinical data in Mayo Clinic primary MM database (n=478). Correlation of PRR11 expression (RNAseq data) with overall survival and number of treatment protocols in a Mayo Clinic MM primary patient dataset (n=487) was analyzed. PRR11 expression increased during sequential phases of treatment and high expression of PRR11 was associated with a shorter survival.

FIGS. 11A and 11B show MM cell growth and drug response after depletion of PRR11 using CRISPR-Cas9 technology or inhibition of PBK in HMCLs. (A) The lentivirus harboring non-targeting control (NS) and two PRR11 CRISPR gRNAs (#1 and #2) expressing cassette were used to infect two MM cell lines (JJN3 and RPMI1640). After confirming depletion of PRR11 in HMCLs by western blot (bottom), an MTT assay was performed to evaluate cell proliferation and drug response to lenalidomide or bortezomib in both control virus-transduced cells and cells with PRR11 depletion. Depletion of PRR11 neither changed MM cell proliferation nor affected lenalidomide and bortezomib sensitivity. (B) JJN3 and XG1LenRes, IMiD resistant cell lines, were treated with a PBK specific inhibitor, HI-TOPK-032 alone (Calbiochem) or combined with either lenalidomide (Len) or bortezomib (Bor) to evaluate synergy by the MTT assay. Inhibition of PBK activity using Hi-TOPK-032, enhanced both lenalidomide and bortezomib sensitivity in JJN3 and XG1LenRes cells.

FIG. 12 shows the heatmap of differentially expressed genes between ND and late/RR samples. 45/121 genes were identified as significantly differentiated between the ND and late/RR samples (P<0.01) using edgeR software. The heatmap view of expression of those differentially expressed genes in ND and RR samples are displayed.

FIGS. 13A and 13B show hierarchical clustering of 45 differentially expressed genes between ND and late/RR samples, and identification of predictive probes. FIG. 13A is a schematic depicting the expression pattern of 45 differentially expressed genes between the ND and late/RR samples (p<0.01), analyzed by Pvclust. Values at branches are AU p-values (red) and BP values (green). Clusters with AU≥90 are indicated by the rectangles. FIG. 13B is a table listing predictive genes identified by analysis of the 45 differentially expressed genes between the ND and late/RR samples (p≤0.01), using single gene glm model regression with coefficient p-value ≤0.05.

FIGS. 14A-14E illustrate the establishment of a predictive model based on the differentially expressed genes between ND and Late/RR samples. FIG. 14A is a table showing a 7-gene predictive model that was built based on a linear logistic regression with R package BhGLM. FIG. 14B is a graph plotting area under the curve (AUC) with 95% confidence interval, resulting from 5-fold cross-validation of established model. FIG. 14C is a graph plotting survival probability. The established model was employed on RNAseq data from the CoMMpass dataset for responder/non-responder prediction. The scores based on the 7-gene expression in each sample were calculated and ranked. The survival data from the 20% of samples that ranked at each side of probability of response were compared, showing that the samples on the non-responder probability side had a shorter survival compared with the samples on the responder probability side.

FIGS. 14D and 14E are graphs plotting responder probabilities determined using the established model on mRNAseq data from the Mayo Clinic MM primary patient dataset. Scores were calculated in the samples grouped by different stage and treatment protocols. These studies showed that patients at the ND stages had a higher probability of being responders than patients during therapy (“other”) or at refractory and end stages (ES) (FIG. 14D). In addition, when patients subjected to varying numbers of treatments (1 or 2 or >3 treatment protocols) were compared, patients with no treatment or fewer treatments had a greater probability of being responders (FIG. 14E).

### DETAILED DESCRIPTION

This document provides methods and materials for identifying and treating mammals (e.g., humans) having MM. For example, this document provides methods and materials for identifying and treating mammals having advanced stage MM, and/or having MM that is resistant to treatment with IMiDs and/or PIs. As described herein, a set of biomarkers associated with IMiD and PI resistance and disease progression in MM was identified using NanoString nCounter technology. NanoString nCounter is a direct multiplexed measurement of gene expression based on digital color-barcoding technology (Kulkarni, Curr Protoc Mot Blot, Chapter 25:Unit 25B 10, 2011), and has been found to be a flexible, reproducible, and robust method in the clinic when used for molecular subtyping of diffuse large B-cell lymphoma (DLBCL) based on the cells of origin (Scott et al., Blood 123(8):1214-1217, 2014; and Veldman-Jones et al., Clin Cancer Res 21(10):2367-2378, 2015). As described herein, NanoString nCounter technology was employed to investigate the transcriptional expression of 121 genes for their association with IMiD or PI response, using 28 human MM cell lines (HMCLs) with known drug sensitivities and 156 MM patient samples collected at different stages of disease, including untreated samples (ND; collected before treatment), samples collected during therapy, and samples from relapsed and refractory disease (RR, collected within 12 months of death). As described herein, it was discovered that a subset of genes were linked to clinical drug resistance, poor survival, and disease progression following treatment with one or more IMIDs and/or one or more PIs.

This document provides methods and materials for identifying and/or treating mammals having, or being likely to have, late stage MM, or mammals having, or being likely to have, refractory/resistant (RR) MM. For example, this document provides methods and materials for identifying a mammal (e.g., a human) as having late stage MM (e.g., end stage MM), or MM that is refractory to treatment with IMiDs and/or PIs. Any appropriate mammal can be identified as having, or being likely to have, late stage MM and/or RR MM as described herein. For example, humans, non-human primates such as monkeys or other mammals (e.g., dogs, cats, horses, cows, pigs, sheep, mice, rabbits, or rats) can be identified as having, or being likely to have, late stage MM and/or RR MM as described herein.

As described herein, a mammal (e.g., a human) can be identified as having, (or being likely to have) advanced stand MM and/or RR MM by determining that a biological sample from the mammal has altered (e.g., elevated or decreased) levels of expression of one or more markers. Examples of markers that can be evaluated and used to classify a mammal (e.g., a human) as having (or being likely to have) advanced stage MM and/or RR MM include, without limitation, CRBN, CEP55, DIRAS1, SKA2, CD53, and PSMD14. Exemplary mRNA sequences for these markers are provided in GENBANK®, as set forth in the table below. The sequences also appear in Appendix A.

| Gene | SEQ ID NO: | Accession No(s). |
|---|---|---|
| CRBN | 1 | NM_001173482.1 |
| | 2 | NM_016302.4 |
| CEP55 | 3 | NM_018131.5 |
| | 4 | NM_001127182.2 |
| DIRAS1 | 5 | NM_145173.4 |
| SKA2 | 6 | NM_182620.4 |
| | 7 | NM_001330399.2 |
| | 8 | NM_001100595.2 |
| CD53 | 9 | NM_001040033.2 |
| | 10 | NM_000560.4 |
| | 11 | NM_001320638.2 |
| PSMA7 | 12 | NM_002792.4 |
| PSMD14 | 13 | NM_005805.6 |

In some embodiments, a marker used in the methods provided herein can have a nucleotide sequence that is at least 90% (e.g., at least 91%, 92%, 93%, 94%, 95%, 96%, 97%, 98%, or 99%) identical to a sequence as set forth in any of SEQ ID NOS:1-13, or to a fragment of a sequence set forth in any of SEQ ID NOS:1-13 (e.g., a fragment that is about 25 to 50 nucleotides in length, 50 to 100 nucleotides in length, 100 to 150 nucleotides in length, or 150 to 200 nucleotides in length).

The percent sequence identity between a particular nucleic acid or amino acid sequence and a sequence referenced by a particular sequence identification number is determined as follows. First, a nucleic acid or amino acid sequence is compared to the sequence set forth in a particular sequence identification number using the BLAST 2 Sequences (B12seq) program from the stand-alone version of BLASTZ containing BLASTN version 2.0.14 and BLASTP version 2.0.14. This stand-alone version of BLASTZ can be obtained online at fr.com/blast or at ncbi.nlm.nih.gov. Instructions explaining how to use the B12seq program can be found in the readme file accompanying BLASTZ. B12seq performs a comparison between two sequences using either the BLASTN or BLASTP algorithm. BLASTN is used to compare nucleic acid sequences, while BLASTP is used to compare amino acid sequences. To compare two nucleic acid sequences, the options are set as follows: -i is set to a file containing the first nucleic acid sequence to be compared (e.g., C:\seq1.txt); -j is set to a file containing the second nucleic acid sequence to be compared (e.g., C:\seq2.txt); -p is set to blastn; -o is set to any desired file name (e.g., C:\output.txt); -q is set to −1; -r is set to 2; and all other options are left at their default setting. For example, the following command can be used to generate an output file containing a comparison between two sequences: C:\B12seq c:\seq1.txt -j c:\seq2.txt -p blastn -o c:\output.txt -q -1 -r 2. To compare two amino acid sequences, the options of B12seq are set as follows: -i is set to a file containing the first amino acid sequence to be compared (e.g., C:\seq1.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\B12seq c:\seq1.txt -j c:\seq2.txt -p blastp -o c:\output.txt. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide residue is presented in both sequences. The percent sequence identity is determined by dividing the number of matches either by the length of the sequence set forth in the identified sequence or by an articulated length (e.g., 100 consecutive bases from a sequence set forth in an identified sequence), followed by multiplying the resulting value by 100. For example, the reference human mRNA sequence set forth in SEQ ID NO:1 is 2593 nucleotides in length. A nucleotide sequence that has 2550 matches when aligned with the reference sequence is 98.3 percent identical to the reference sequence (i.e., 2550/2593×100=98.3). It is noted that the percent sequence identity value is rounded to the nearest tenth. For example, 75.11, 75.12, 75.13, and 75.14 are rounded down to 75.1, while 75.15, 75.16, 7.17, 75.18, and 7.19 are rounded up to 7.2. It also is noted that the length value will always be an integer.

Any appropriate biological sample can be used in the methods provided herein. Suitable biological samples include, without limitation, (e.g., blood and bone marrow). Any appropriate method can be used to determine if a mammal (e.g., a human) has an altered (e.g., elevated or reduced) level of one or more markers described herein. The term “elevated level” or “increased level” as used herein with respect to a marker level refers to a level (e.g., an mRNA level or a polypeptide level) of the marker in a biological sample that is greater (e.g., at least 5, 10, 25, 35, 45, 50, 55, 65, 75, 80, 90, 100, or more than 100 percent greater) than the median level of that marker in a control biological sample from a control mammal (e.g., a healthy mammal that does not have MM, or a mammal that does not have advanced stage or RR MM). The term “reduced level” or “decreased level” as used herein with respect to a marker level refers to a level (e.g., an mRNA level or a polypeptide level) of the marker in a biological sample that is less (e.g., at least 5, 10, 25, 35, 45, 50, 55, 65, 75, 80, 90, or 100 percent less) than the median level of that marker in a control biological sample from a control mammal (e.g., a healthy mammal that does not have MM, or a mammal that does not have advanced stage MM or RR MM). Appropriate methods for identifying a biological sample as having an elevated or reduced level of one or more markers described herein include, without limitation, mRNA assessment techniques such as NanoString technology, real-time quantitative polymerase chain reaction (RT-qPCR), northern blotting, or RNA sequencing and microarray expression profiling. In some cases, appropriate methods for identifying a biological sample as having an elevated or reduced level of one or more markers described herein include polypeptide assessment techniques such as immunohistochemistry and enzyme-linked immunosorbent assays (ELISA).

Once a mammal (e.g., a human) is identified as having a biological sample with altered (e.g., elevated or reduced) levels of one or more of the markers described herein, the mammal can be classified as having, or being likely to have, advanced stage MM and/or RR MM. For example, a human identified as having a biological sample with an altered expression level of one or more (e.g., two, three, four, five, six, or all seven) markers selected from CRBN, CEP55, DIRAS1, SKA2, CD53, PSMA7, and PSMD14 can be classified as having, or being likely to have, advanced stage MM and/or RR MM. In some cases, a mammal (e.g., a human) identified as having a biological sample that does not exhibit an altered expression level of CRBN, CEP55, DIRAS1, SKA2, CD53, PSMA7, and PSMD14 can be classified as not having, or not being likely to have, advanced MM or RR MM.

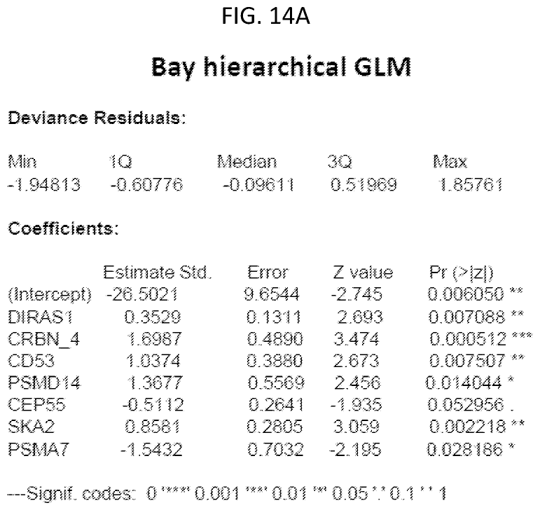

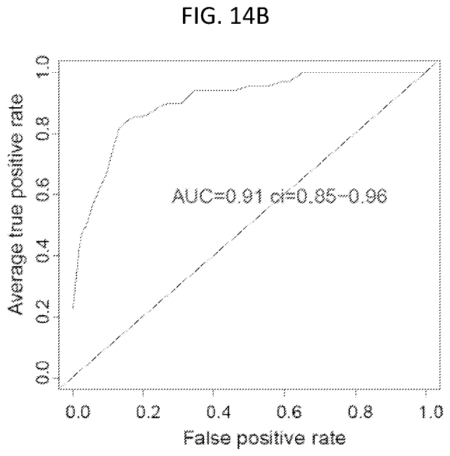

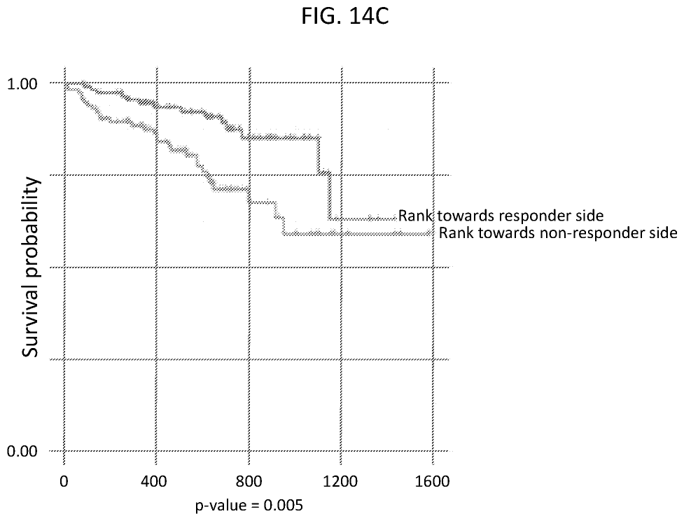

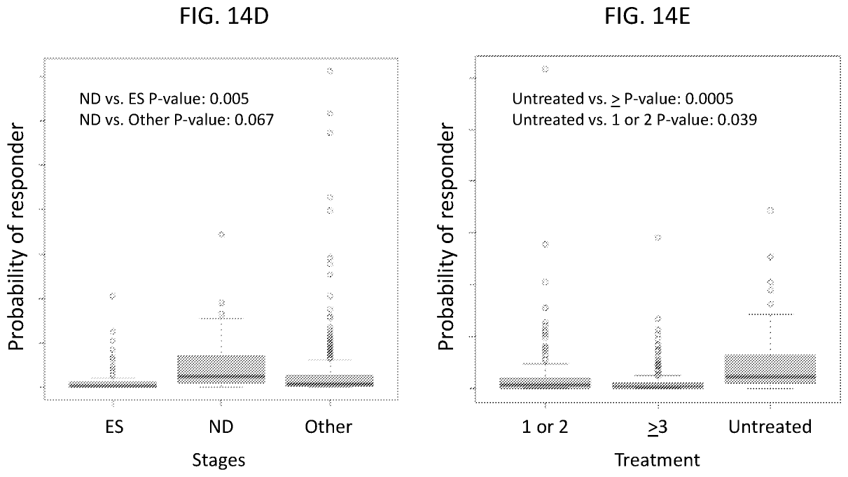

Any combination of the markers disclosed herein can be evaluated. For example, when altered levels of two markers are used to identify a mammal as having, or being likely to have, advanced stage MM and/or RR MM, the two markers can be CRBN and CEP55, CRBN and DIRAS1, CRBN and SKA2, CRBN and CD53, CRBN and PSMA7, CRBN and PSMD14, CEP55 and DIRAS1, CEP55 and SKA2, CEP55 and CD53, CEP55 and PSMA7, CEP55 and PSMD14, DIRAS1 and SKA2, DIRAS1 and CD53, DIRAS1 and PSMA7, DIRAS1 and PSMD14, SKA2 and CD53, SKA2 and PSMA7, SKA2 and PSMD14, CD53 and PSMA7, CD53 and PSMD14, or PSMA7 and PSMD14. Similarly, any combination of three, four, five, six, or all seven of the aforementioned markers can be evaluated to determine whether a mammal has, or is likely to have, advanced stage MM and/or RR MM. In some cases, the probability of being a responder can be calculated by based on the expression of all seven genes (e.g., based on the ordinal model depicted in FIG. 14A). Gene expression levels positively related to the probability of being a responder show positive coefficients in the model, and vice versa.

In some cases, a method provided herein can include identifying a mammal as having, or being likely to have, advanced MM and/or RR MM when a biological sample from the mammal is determined to exhibit reduced expression of one or more of CRBN, DIRAS1, CD53, SKA2, and/or elevated expression of one or more of CEP55, PSMA7 and PSMD14. In some cases, a method provided herein can include identifying a mammal as having, or being likely to have, advanced MM and/or RR MM when a biological sample from the mammal is determined to exhibit reduced expression of CRBN, DIRAS1, CD53, and SKA2, and elevated expression of CEP55 and PSMD14.

This document also provides methods and materials for treating a mammal identified as having, or as being likely to have, advanced MM and/or RR MM. Any appropriate mammal identified as having, or as being likely to have, advanced MM and/or RR MM can be treated with anti-MM agents such as, for example, a daratumumab based regimen such as DPd (daratumumab, pomalidomide, and dexamethasone), chimeric antigen receptor T cells (CAR-T cells) against a target such as, without limitation, BCMA, GPCR5, or FCRH5, histone deacetylase (HDAC) inhibitors, or panobinostate-based therapies (e.g., PI and panobinostat). Having the ability to identify mammals who have or are likely to have advanced stage and/or RR MM can allow clinicians and patients to proceed with treatment options that more effectively treat the MM (e.g., to achieve better disease control). In addition, mammals identified as having, or being likely to have, advanced MM and/or RR MM can undergo more regular surveillance via, for example, X-rays, positron emission tomography (PET) scans, magnetic resonance imaging (MM), bone density scans, or computed tomography (CT) scans to detect changes in MM status and response to treatment.

In some cases, an effective dose of one or more therapies (e.g., DPd, CAR-T cells, or HDAC inhibitors) can be administered to a mammal once or multiple times over a period of time ranging from days to months. Effective doses can vary depending on the severity of the MM, the route of administration, the age and general health condition of the subject, excipient usage, the possibility of co-usage with other therapeutic treatments, and the judgment of the treating physician.

An effective amount of a composition (e.g., a composition containing DPd, CAR-T cells or HDAC inhibitors) can be any amount that reduces the likelihood that the MM will progress, or any amount that reduces disease symptoms, or any amount that prolongs survival (e.g., overall survival or progression-free survival) without producing significant toxicity to the mammal. For example, an effective amount of daratumumab can be from about 16 mg/kg/week to about 16 mg/kg/month (e.g., from about 4 mg/kg/week to about 8 mg/kg/week, from about 8 mg/kg/week to about 12 mg/kg/week, or from about 12 mg/kg/week to about 16 mg/kg/week), and an effective amount of dexamethasone can be from about 10 mg/week to about 80 mg/week (e.g., from about 10 mg/week to about 20 mg/week, from about 20 mg/week to about 40 mg/week, or from about 40 mg/week to about 60 mg/week).

The frequency of administration of a MM treatment (e.g., DPd, CAR-T cells, or HDAC inhibitors) to a mammal can be any frequency that reduces the symptoms of the MM, reduces the likelihood that the MM will progress, or increases survival (e.g., overall survival or progression-free survival) of the mammal without producing significant toxicity to the mammal. For example, the frequency of administration can be from about once a day to about once a month (e.g., from about once a week to about once every other week). The frequency of administration can remain constant or can be variable during the duration of treatment. A course of treatment with a composition containing one or more agents (e.g., DPd, CAR-T cells, or HDAC inhibitors) can include rest periods. For example, a composition can be administered daily over a two-week period followed by a two-week rest period, and such a regimen can be repeated multiple times. As with the effective amount, various factors can influence the actual frequency of administration used for a particular application. For example, the effective amount, duration of treatment, use of multiple treatment agents, route of administration, and severity of the condition may require an increase or decrease in administration frequency.

An effective duration for administering a composition containing one or more agents for treating MM (e.g., CAR-T cells or HDAC inhibitors) to a mammal can be any duration that alleviates one or more symptoms of the MM, reduces the likelihood that the MM will progress, or increases survival (e.g., overall survival or progression-free survival) of the mammal, without producing significant toxicity to the mammal. In some cases, the effective duration can vary from months to years. Multiple factors can influence the actual effective duration used for a particular treatment. For example, an effective duration can vary with the frequency of administration, effective amount, use of multiple treatment agents, route of administration, and severity of the condition being treated.

The invention will be further described in the following examples, which do not limit the scope of the invention described in the claims.

### EXAMPLES

### Example 1—Material and Methods

Studies were conducted to investigate the transcriptional expression of 121 genes linked to drug resistance, taken from an in-house database and described elsewhere (see, e.g., Zhu et al., 2011, supra; Kronke et al., supra; Zhu et al., 2019, supra; Eichner et al., Nat Med 22(7):735-743, 2016; Daniel et al., Cancer Res 79, 2019; Zhu et al., Blood 124(4):536-545, 2014; An et al., Nat Commun 8:15398, 2017; Chapman et al., Blood 132(20):2154-2165, 2018; Pelham et al., Blood 130(Suppl 1):839, 2017; Mitra et al., Leukemia 30(5):1094-1102, 2016; and Shi et al., Blood 134(Suppl. 1):4337, 2019). These studies included screening of 28 HMCLs with known drug sensitivities and 156 primary MM patient samples collected at various stages of disease evolution, including newly diagnosed, during treatment, and in late relapse within 12 months of demise. Gene expression profiles between sample groups with expected distinct drug response profiles and disease stages (sensitive versus resistant HMCLs and newly diagnosed versus during treatment, or versus late or relapsed and refractory, patient samples) were analyzed and compared.

Collated gene list and test samples used for NanoString profiling: A unique list was selected to include 64 IMiD and 57 PI resistance associated gene candidates; this list was used to generate CodeSets for NanoString profiling (TABLE 1). Initial selections included 26 probes targeting CRBN, genes with altered transcription in cell lines with low versus normal CRBN, genes encoding proteins associated with CRBN activity (Eichner et al., supra), or genes known to be linked to IMiDs activity and sensitivity (such as IKZF1, IKZF3, IRF4 and MYC). Since CRBN isoforms, including the isoform lacking exon 10, have been associated with IMiD sensitivity (Maity et al., Blood 124(21):639, 2014), four probes targeting different exon junctions of CRBN were included. One (CRBN 3) was designed to span the exon 10/11 junction. Twenty-one (21) genes were selected by analyzing baseline gene expression levels associated with drug response in a cohort of 44 refractory MM patients before initiation of pomalidomide and dexamethasone therapy on a phase 2 clinical trial (Zhu 2014, supra; Lacy et al., J Clin Oncol 27(30):5008-5014, 2009; and Lacy et al., Leukemia 24(11):1934-1939, 2010) and from the isogenic lenalidomide sensitive/resistant HMCL XG1 pair (XG1/XG1LenRes) with normal CRBN levels (Geo123506) (Zhu et al. 2019, supra). Twenty (20) of these gene targets were identified between the responders and non-responders in both data sets. Ten genes included in the panel were selected based on data from 59 MM patients that exhibited differential responses to a first line of treatment containing IMiDs in the CoMMpass data, generated as part of the Multiple Myeloma Research Foundation Personalized Medicine Initiatives). These 10 genes were differentially expressed in samples that showed complete response (CR), partial response (PR) and stable disease (SD), five of which were also identified from the XG1/XG1LenRes data. In addition, 24 genes whose expression was noted elsewhere to predict response to IMiDs and/or PIs in MM (Chapman et al., supra; Pelham et al., supra; and Mitra et al., supra) were included. Finally, 40 gene probes encoding multiple proteasome subunits (A, B, C, and D) were included; these were upregulated in PI resistant cell lines when compared with their isogenic sensitive cell lines (Shi et al., supra).

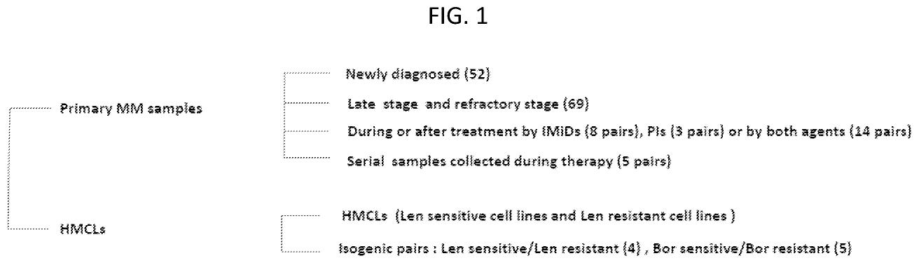

One hundred fifty-six (156) primary MM patient samples and 28 HMCLs (FIG. 1) were used for these studies. Patient samples were divided into several groups based on the time at which samples were collected. These included paired or serial samples in 51 patients. The HMCLs included drug sensitive HMCLs, as well as HMCLs with intrinsic and acquired resistance.

MM cell lines and human MM cells: All HMCLs were fingerprinted to confirm their identity (Keats et al., Blood 110(11):2485, 2007). The cells were cultured in RPMI1640 medium supplemented with 5% fetal calf serum. Isogenic IMID and PI sensitive and resistant cell lines were generated as described elsewhere (Zhu et al. 2019, supra; and Shi et al., supra). The generation of OCIMY5/Vec and OCIMY5/CRBN also was as described elsewhere (Zhu et al. 2014, supra).

Primary human MM cells were recovered from bone marrow aspirates. After collection, CD138+ cells were isolated by immunomagnetic bead selection (RoboSep; Stemcell Technologies).

Sample preparation and RNA extraction: Total RNA from all cell lines and primary patient samples was isolated using the RNEASY® Mini kit and the ALLPREP® DNA/RNA Kit (Qiagen; Hilden, Germany) respectively. After spectrophotometric quantification using a NANODROP™ 2000 (Thermo Fisher Scientific; Waltham, MA), samples were stored at −80° C.

NanoString CodeSet design and expression quantification: One hundred twenty-one (121) genes potentially associated with IMiD and PI response were selected, along with 11 housekeeping genes (TABLE 1), in order to generate the CodeSet for this study. The target-specific oligo probes were designed by NanoString Technologies (Seattle, WA) and synthesized by IDT (Coralville, Iowa). The experiments were performed with nCounter elements XT reagents in accordance with the manufacturer's recommendations. An input of 100 ng total RNA was used for both HMCLs and primary patient samples. The collected data was first evaluated for quality control, followed by technical normalization using synthetic controls and biological normalization via housekeeping genes. Samples that failed to pass quality control or that were flagged after either normalization step were removed. Data generated from remaining samples were analyzed using nSolver 4.0 software and an advanced analysis software plugin (version 2.0, R-based statistical tool) to detect and visualize differentially expressed genes.

Analysis of the survival data in two large MM sequencing datasets: Several genes identified as differentially expressed between ND and late/RR stage patient samples were further analyzed in order to verify correlations between their expression (RNAseq data) with progression free survival (PFS), overall survival (OS), and drug response duration in the MMRF CoMMpass database. Correlation of PRR11 expression (RNAseq data) with OS and number of treatment protocols was also analyzed in a MM patient dataset (n=487).

Analysis of NM cell proliferation and drug response after modulating PRR11 expression and PBK1 activity: PRR11 and PBK1 expression also was associated with survival and drug response in the CoMMpass dataset. Using CRISPR-Cas9 technology, MM cell proliferation and drug response were investigated after depletion of PRR11. Briefly, the lentivirus harboring non-targeting control and two PRR11 CRISPR gRNA expressing cassettes were prepared and used to infect MM cell lines using methods described elsewhere (Ran et al., Nat Protoc 8(11):2281-2308, 2013; and Zhu et al., Cancer Res 77(16):4317-4327, 2017). After confirming depletion of PRR11 in HMCLs by western blot, an MTT assay was performed to evaluate cell proliferation and drug response to lenalidomide or bortezomib in both control virus-transduced cells and cells with PRR11 depletion. For PBK, JJN3 and XG1LenRes (IMiD resistant cell lines) were treated with a PBK specific inhibitor (HI-TOPK-032; Calbiochem; San Diego, CA) either alone or in combination with either lenalidomide or bortezomib to evaluate their effects by MTT assay.

Ranking predictive probes and development of models for predicting drug sensitivity and disease progression: Differentially expressed genes between ND (n=52) and late/RR samples (n=69) were selected using edgeR (Robinson et al., Bioinformatics 26(1):139-140, 2010). The expression pattern of differentially expressed genes was analyzed by supervised Pvclust (Suzuki and Shimodaira, Bioinformatics 22(12):1540-1542, 2006). Each gene was then individually analyzed using a generalized linear model (Yi et al., Bioinformatics 35(8):1419-1421, 2019) to filter the top q-associated probes with different response outcomes. To build a multivariate ordinal model for prediction with the 121 gene probes and annotated ND or RR for each patient sample, a linear logistic regression model was built using R package bhGLM (Yi et al., supra), followed by step AIC (Zhang, Ann Transl Med 4(7):136, 2016) for optimization (removing the non-significant genes).

Assessment of performance of established model: Probabilities were estimated for the 7-gene model. In particular, the performance of the model was evaluated by 5-fold cross-validation as described elsewhere (rdrr.io/cran/cvAUC/man/ci.cvAUC.html). The model also was employed to analyze RNAseq data from the MMRF coMMpass (n=578, newly diagnosed patients) and Mayo Clinic MM primary patients (n=487, collected from patients at different disease stages) datasets to look at the relevance of estimation from this model to other clinical data such as survival, disease stage, and number of treatment protocols. Briefly, RNAseq data was run on this model to calculate probabilities (by ranking scores) and then compared estimated results with other clinical data in each dataset. Since the RNAseq data has different scales when compared to NanoString, the probability of estimate from this analysis is based on ranking order rather than actual criteria.

### Example 2—Validation and Quality Control of NanoString Expression Profiling in MM

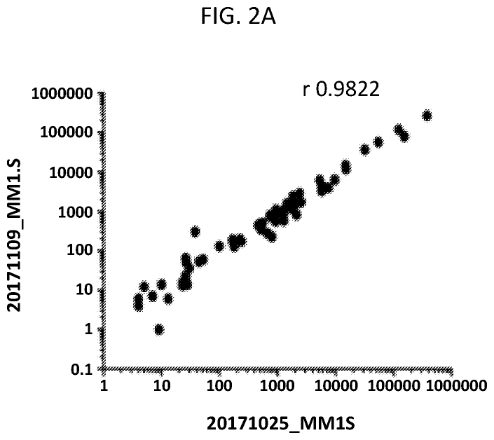

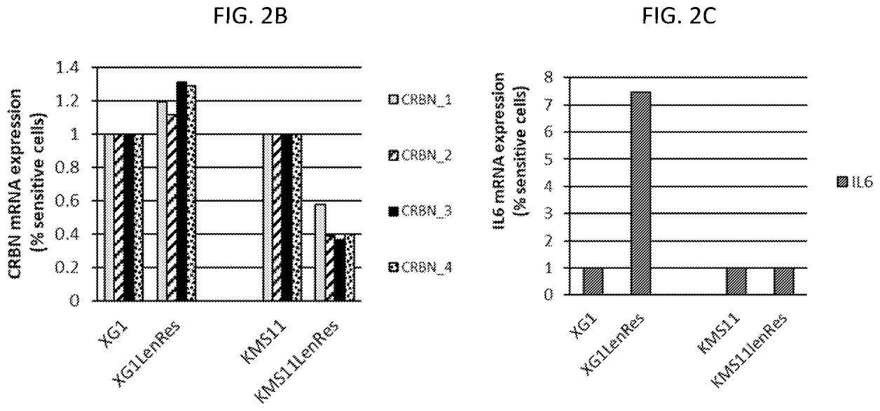

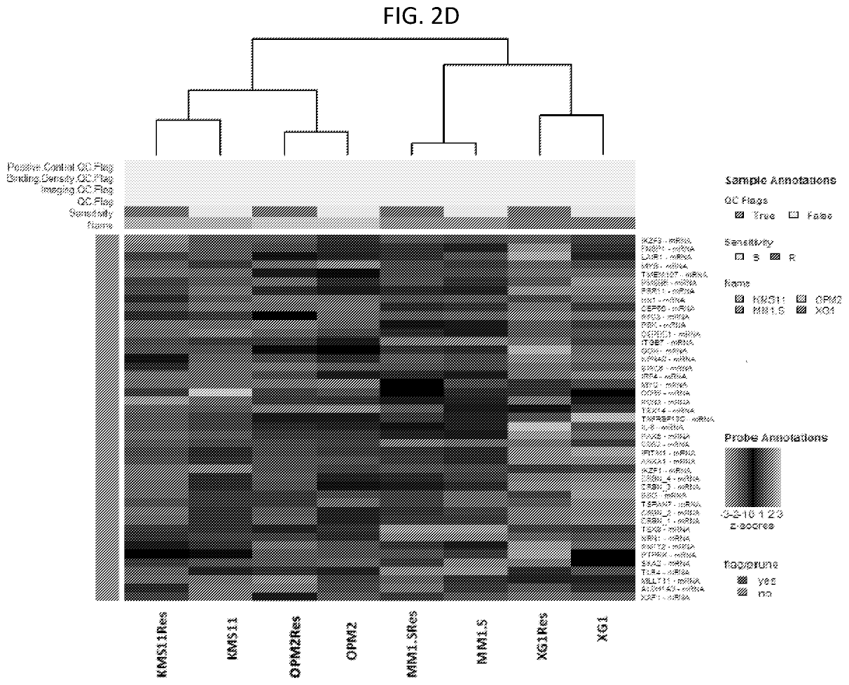

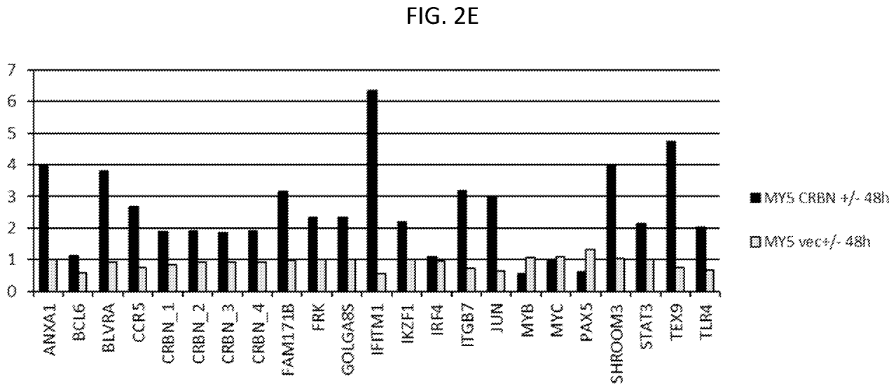

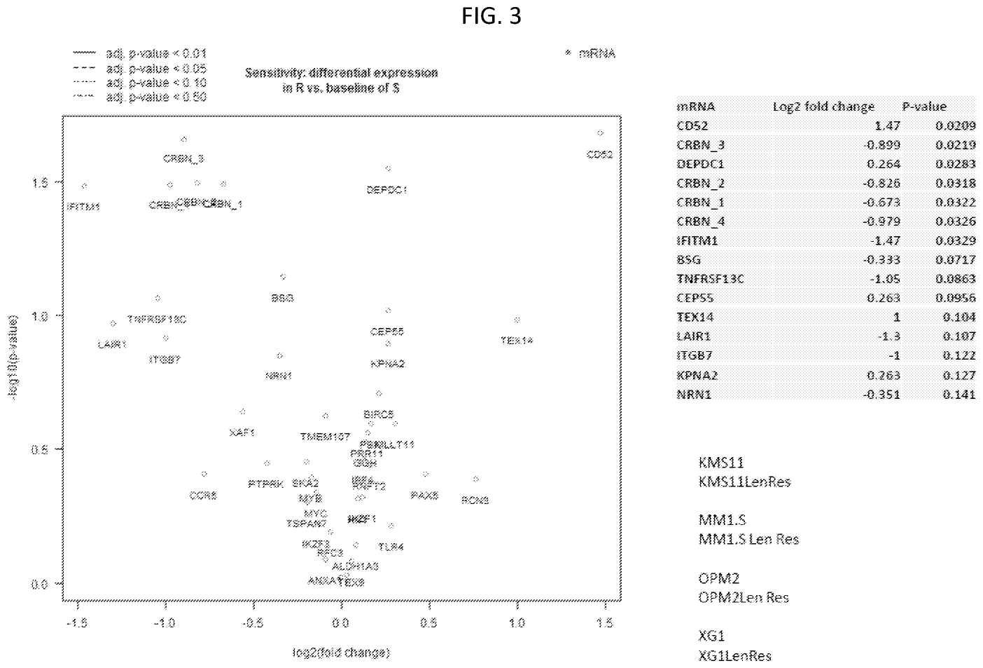

The working conditions for NanoString profiling of MM cells were established by testing a CodeSet of 48 genes (TABLE 1), demonstrating that nCounter technology is able to generate reproducible results from two biological repeats (MM1.S, FIG. 2A). The NanoString assay also detected known CRBN downregulation and IL6 up-regulation in two established lenalidomide resistant HMCLs (compared with their isogenic sensitive cell lines, FIGS. 2B and 2C), consistent with observations described elsewhere (Zhu et al. 2019, supra). A gene expression heatmap of normalized data from four pairs of lenalidomide isogenic HMCLs showed that each isogenic cell line pair clustered together as expected. Further analysis of the expression data using the nSolver 4.0 software also identified downregulation of CRBN as a significant change in those three resistant cell lines (FIGS. 2D and 3).

Using the complete CodeSet, the relative expression levels of the 121 selected genes in OCIMY5/vec (which expresses only a very small amount of CRBN and is resistant to IMiDs) and OCIMY5/CRBN (which has enforced CRBN expression and is sensitive to IMiDs) were measured. Known lenalidomide-regulated gene expression changes were accurately detected (FIG. 2E). Therefore, NanoString technology was confirmed as a sensitive, reliable and reproducible method to quantitate changes in gene expression of MM cells.

### Example 3—Identification of Differentially Expressed Genes in Drug Resistant Patients

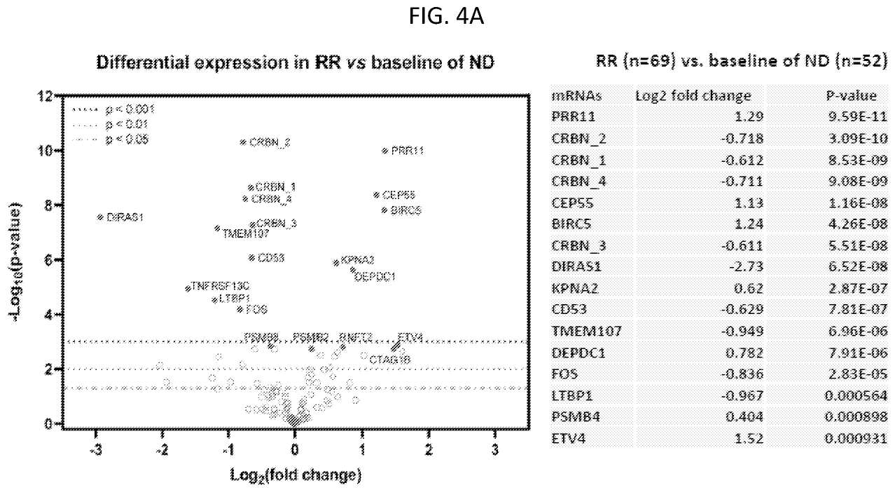

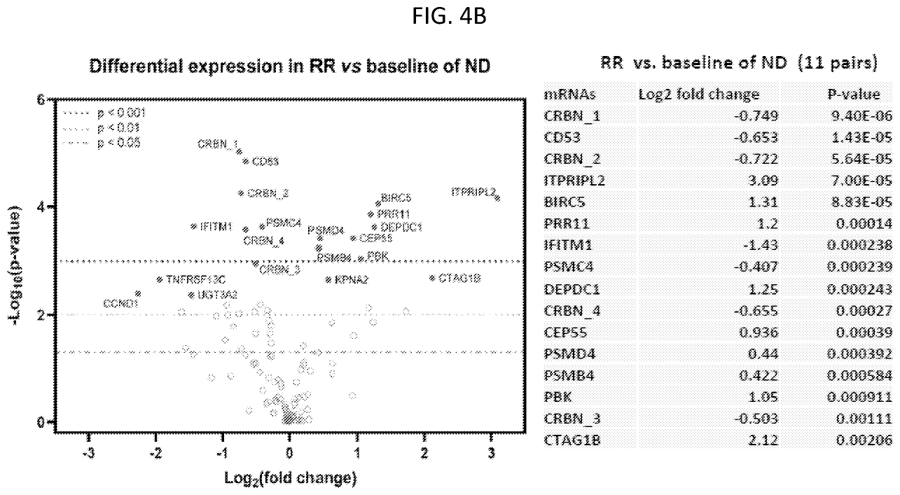

Differential expression of the 121 genes was then measured in all primary MM samples and HMLCs, grouped by known or likely drug sensitivity and resistance profiles. Forty three genes were identified to have a significantly differential expression (p≤0.05) between 52 newly diagnosed and 69 late stage or relapsed refractory samples (FIG. 4A). In addition to the expected CRBN, 6 genes (TMEM107, DIRAS1, CD53, TNFRSF13C, LTBP1, and FOS) were identified as being most significantly downregulated in relapse and RR samples, while another 7 genes (PRR11, CEP55, BIRC5, KPNA2, DEPDC1, PSMB4, and ETV4) were identified as being most significantly upregulated (FIG. 4A).

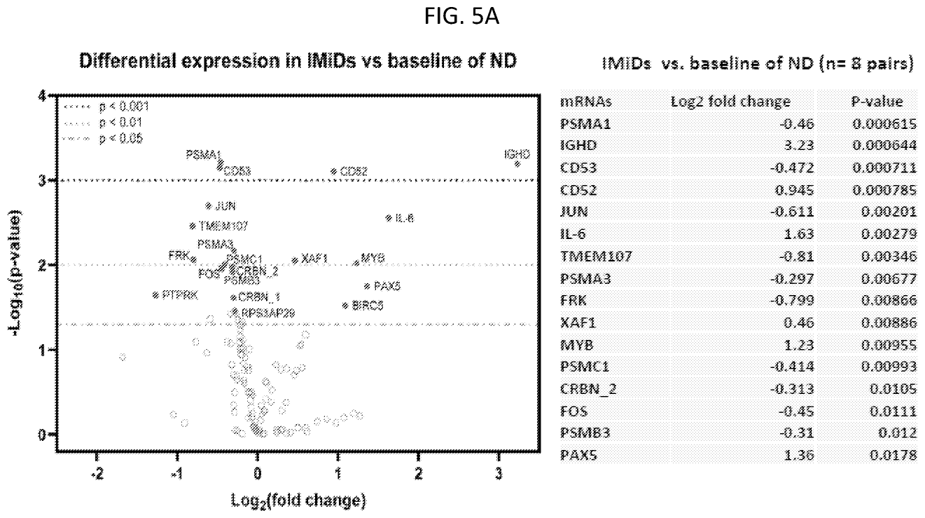

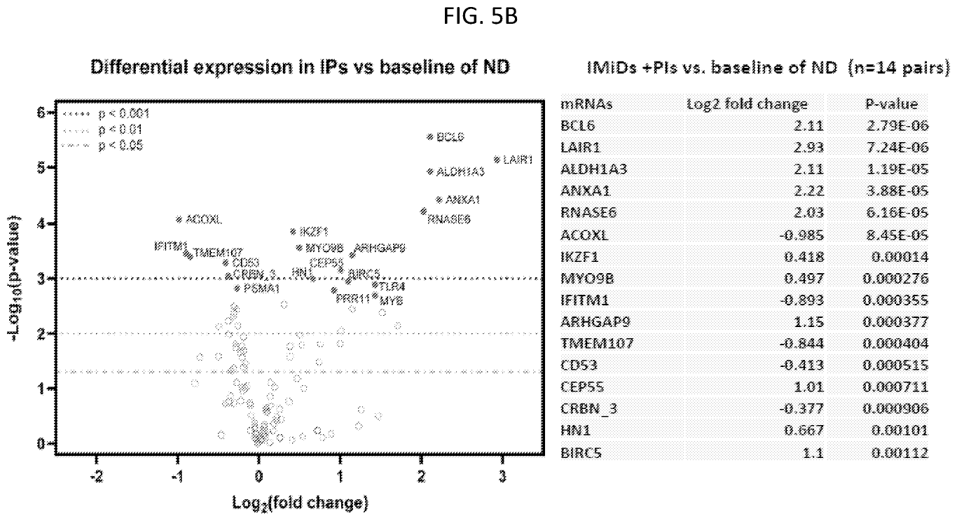

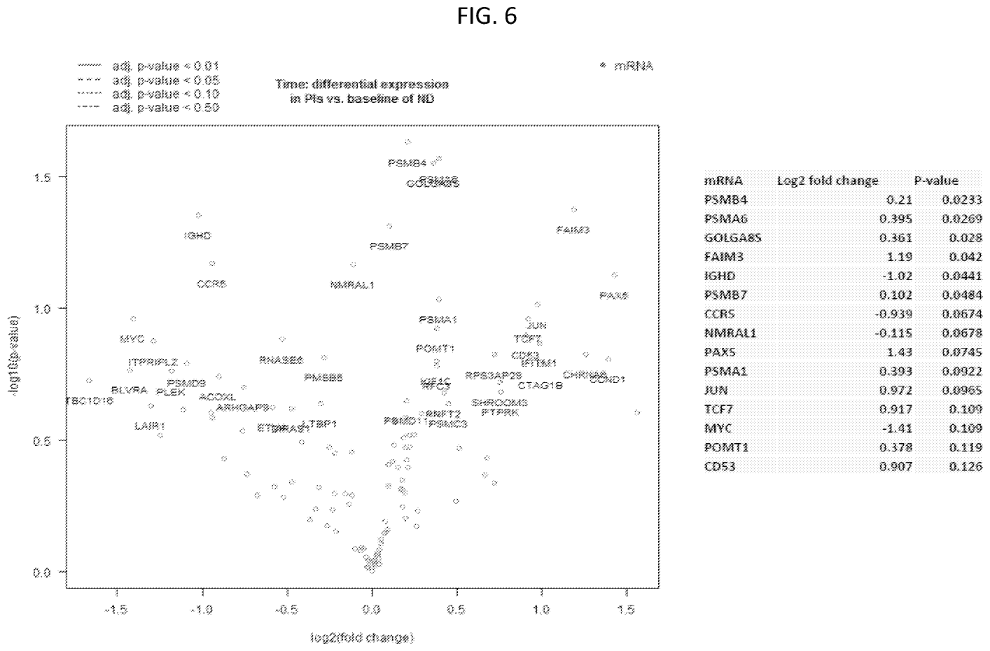

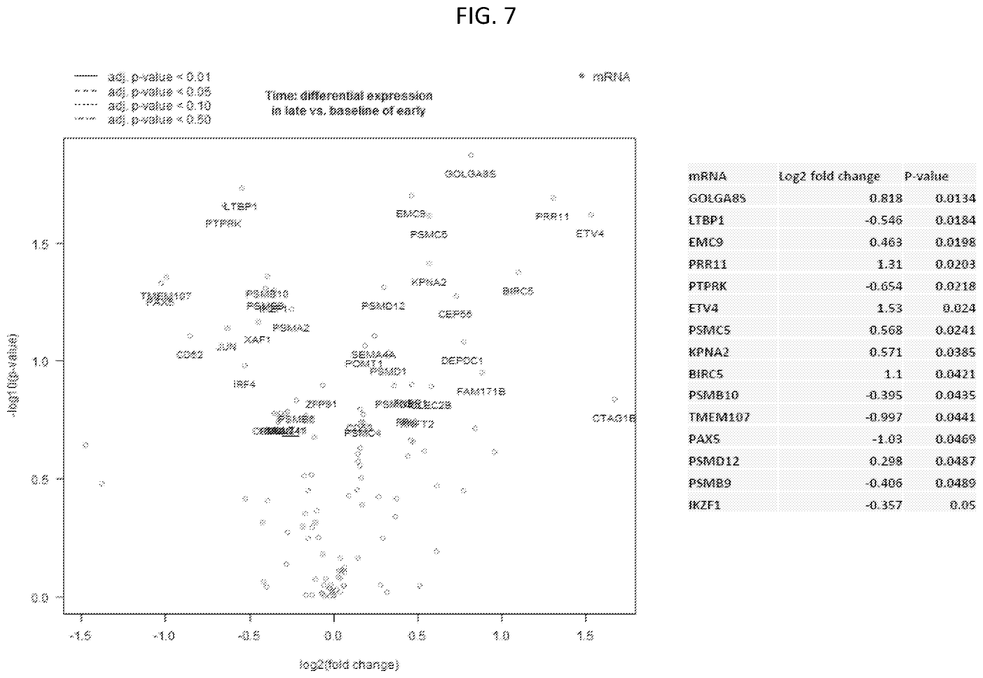

Twenty-two (22) paired-samples from 11 patients, which were collected at both ND and RR stages were then analyzed. Forty-five (45) genes were identified as being differentially expressed between ND and RR stages (p≤0.05). In addition to confirming the transcriptional changes described above, the RR samples also showed downregulation of IFIRM1 and PSMC4, and upregulation of ITPRIPL2, PBK, PSMD4, and CTAG1B in their most differentially expressed genes (FIG. 4B). These changes were not detected, or were detected at lower significance, when comparing paired samples at ND with a secondary sample collected during treatment but before disease progression; this dataset included samples from patients treated with IMiDs (8 pairs, FIG. 5A), IMiDs+PI (14 pairs, FIG. 5B), and solely PIs (3 pairs, FIG. 6). When comparing ND samples with paired “on active treatment” samples, downregulation of CRBN and CD53 and upregulation of PRR11, CEP55, and BIRC5 was demonstrated (FIG. 5B). A similar trend of upregulation of PRR11, ETV4, and BIRC5 was also identified in later relapse samples compared with early samples collected during treatment from 5 patients (FIG. 7).

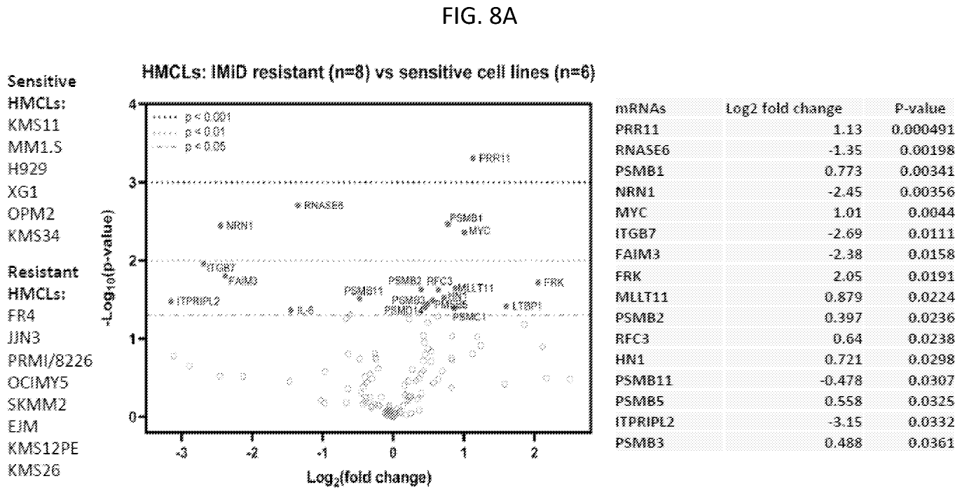

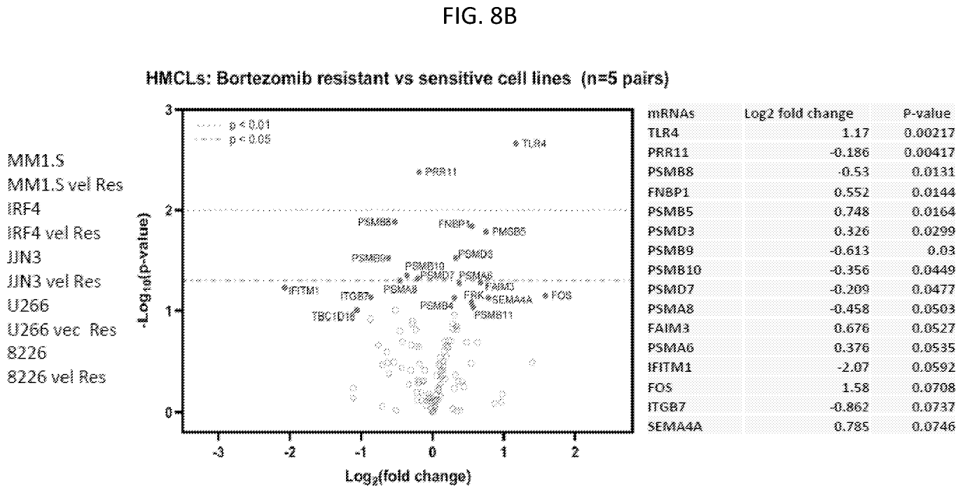

HMCLs with known responses to IMiDs and PIs (de Campos et al., Blood Cancer J 10:54, 2020) were then evaluated. By comparing gene expression of six WED-sensitive and eight IMiD-resistant HMCLs, 22 genes were identified as differentially-expressed (FIG. 8A). Six changes in WED resistant HMCLs were consistent with those identified in RR samples from MM patients, including upregulation of PRR11, HN1, RFC3, PSMB2, and PSMD14, and downregulation of SKA2. When five PI sensitive and resistant isogenic cells lines were compared, changes in the expression of seven proteasome subunit genes were identified, including upregulation of PSMB5 in resistant cell lines (FIG. 8B).

### Example 4—Clinical and Functional Analysis of Top Identified Genes

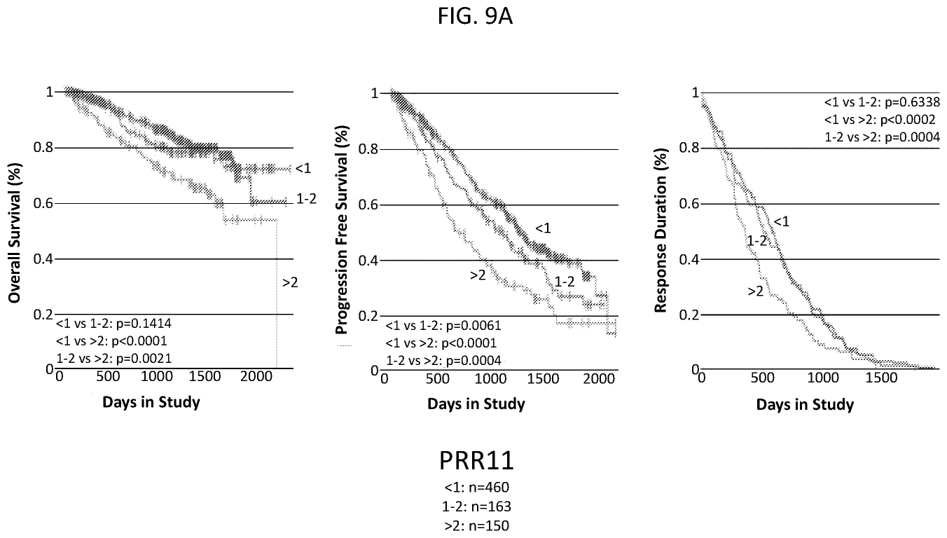

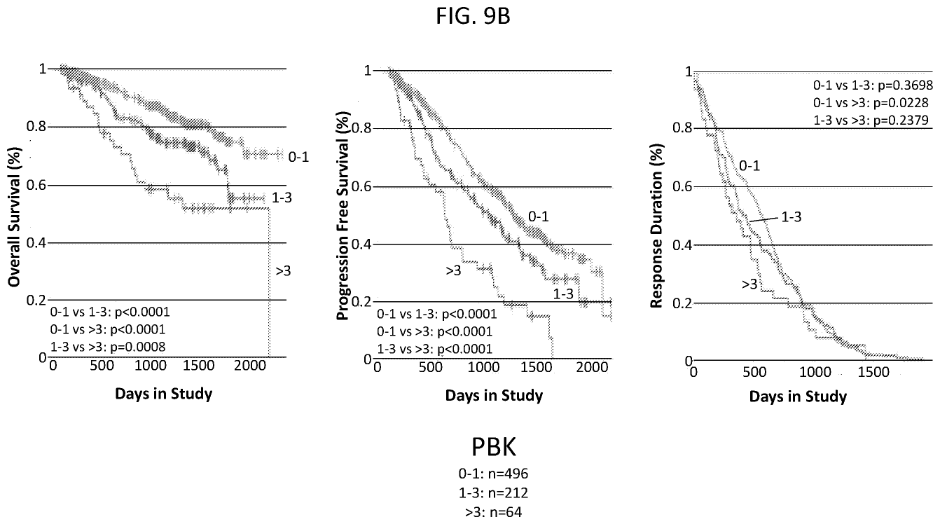

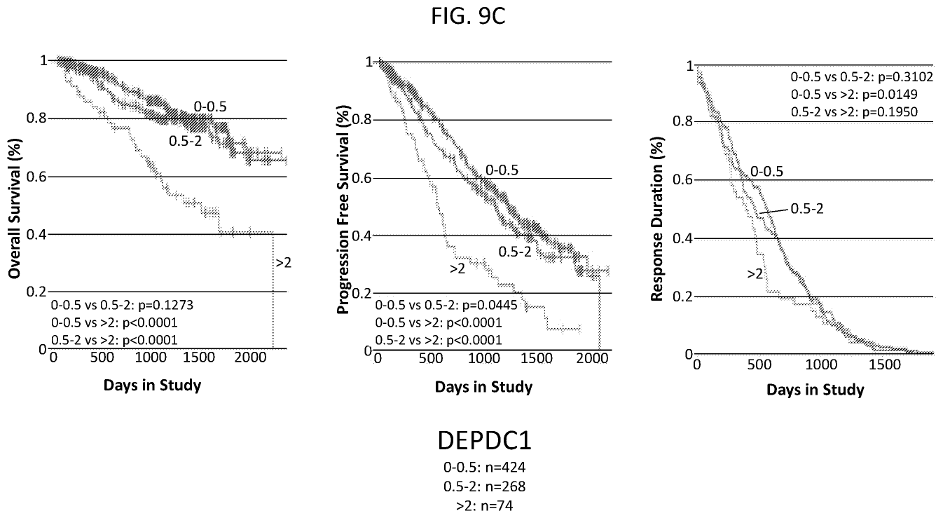

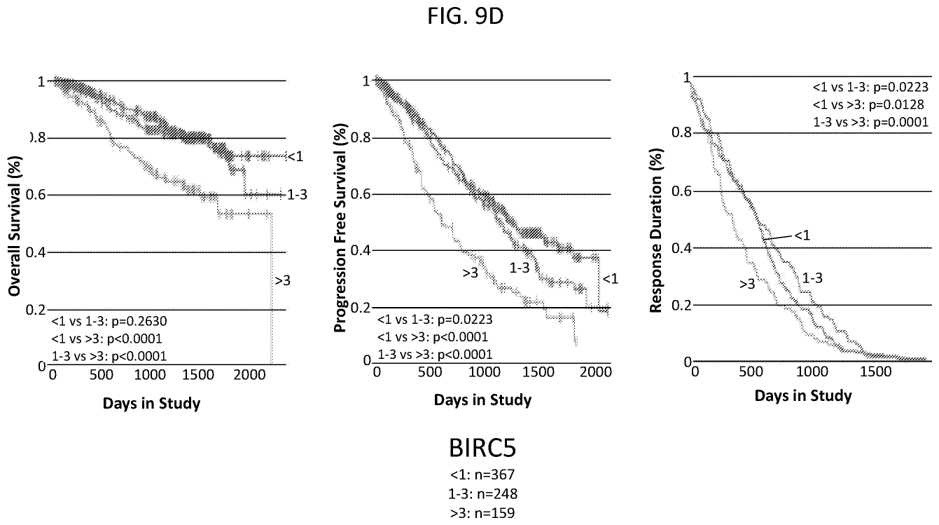

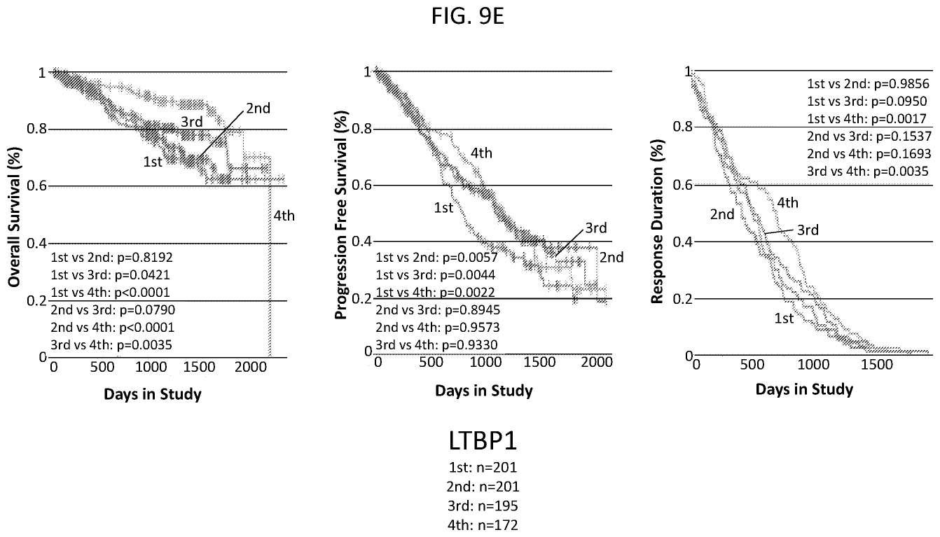

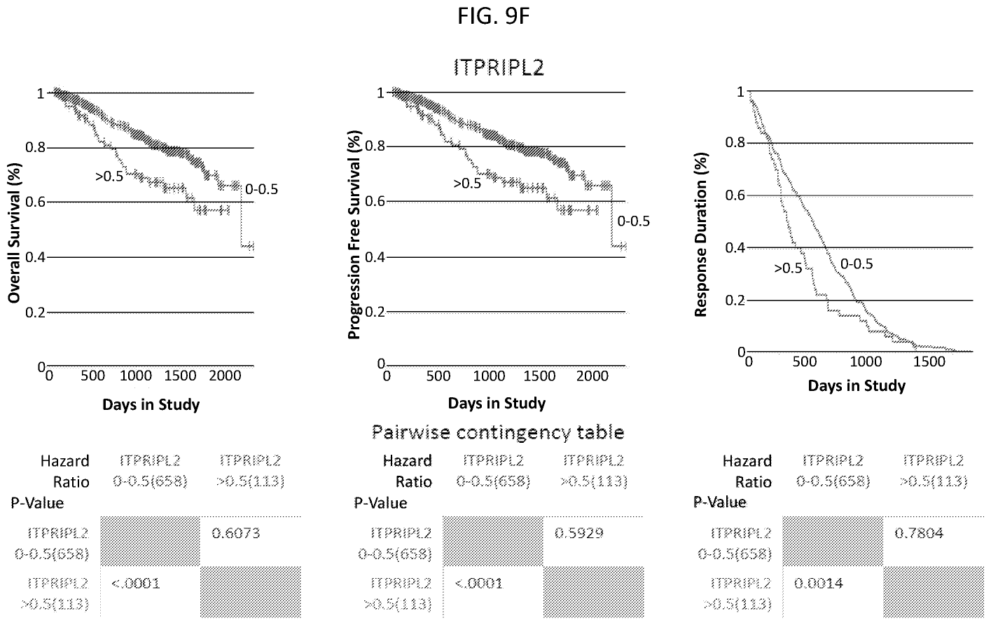

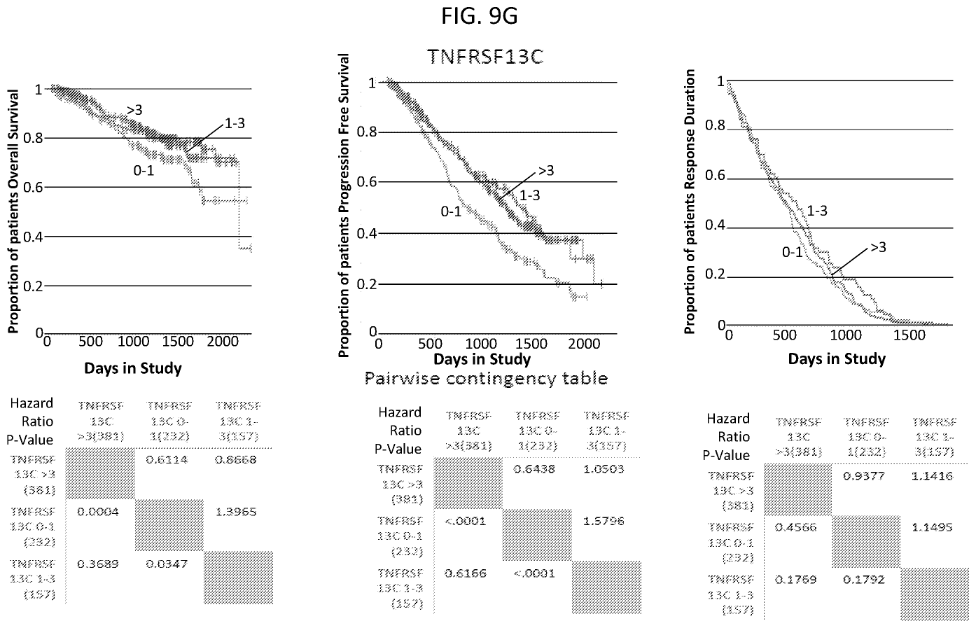

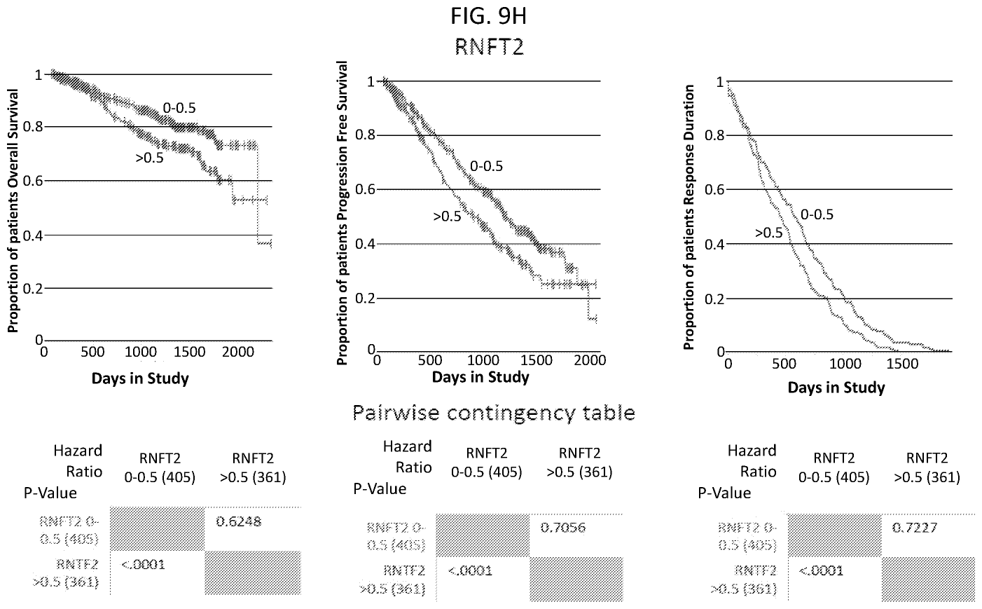

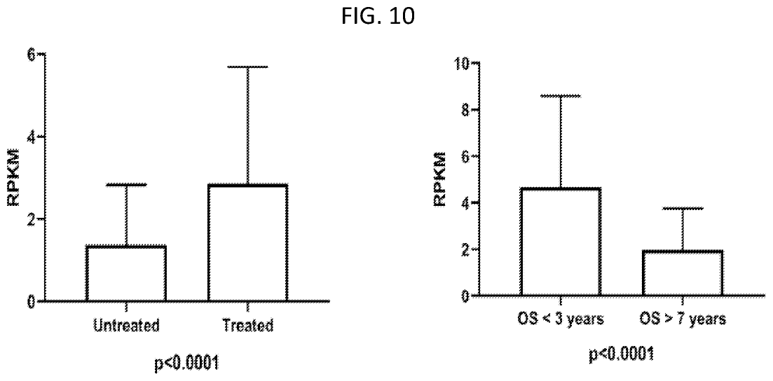

The relevance of the above findings in patient survival and drug responses was evaluated through the analysis of the expression of significant markers (n=8) of early or late stage disease using the MMRF CoMMpass database. As shown in FIGS. 9A-9H, samples with high expression of PRR11, PBK, DEPDC1, BIRC5, RNFT2, and ITPRIPL2 in the CoMMpass data correlated with shorter OS, shorter PFS, and/or poor drug response, whereas samples with high expression of LTBP1 and TNFRSF13C were associated with a longer OS, longer PFS, and/or improved response to therapy. Considering that PRR11 was upregulated in both HMCLs and in resistant patient groups, gene expression was further examined in the Mayo Clinic dataset (n=487). As in the CoMMpass and NanoString analysis, PRR11 expression was increased during sequential phases of treatment, and high expression of PRR11 was associated with a shorter survival (FIG. 10).

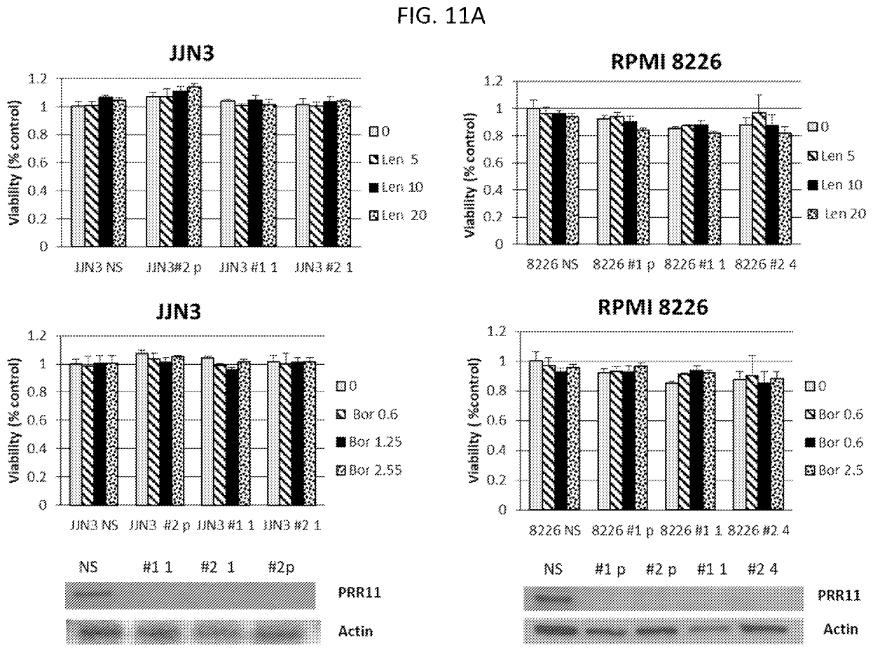

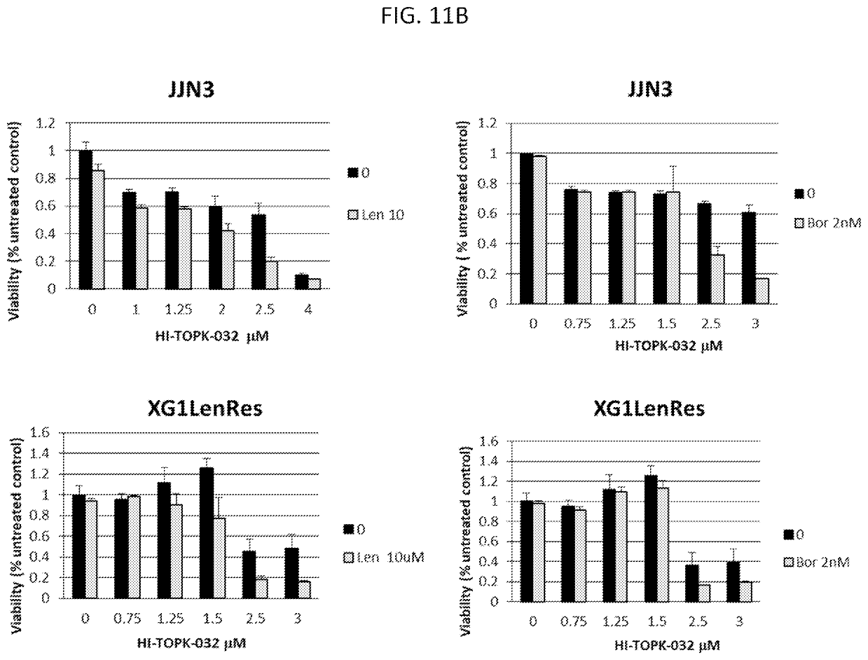

To understand whether PRR11 is important for MM growth and is directly involved in the response to IMiDs and PIs, CRISPR-Cas9 technology was used to deplete PRR11 in two HMCLs, and the effect on cell proliferation and drug response was tested. Depletion of PRR11 did not change MM cell proliferation, nor did it affect IMiD and PI sensitivity (FIG. 11A), suggesting that PRR11 is not directly involved in MM cell proliferation and drug response. An additional consistently upregulated gene in late stage disease, PBK, was then studied using a specific PBK inhibitor (Hi-TOPK-032). These studies showed that Hi-TOPK-032 inhibition of PBK activity enhanced both lenalidomide and bortezomib sensitivity in JJN3 and XG1LenRes cells (FIG. 11B).

### Example 5—Identifying Predictive Probes and Establishing a Predictive Model

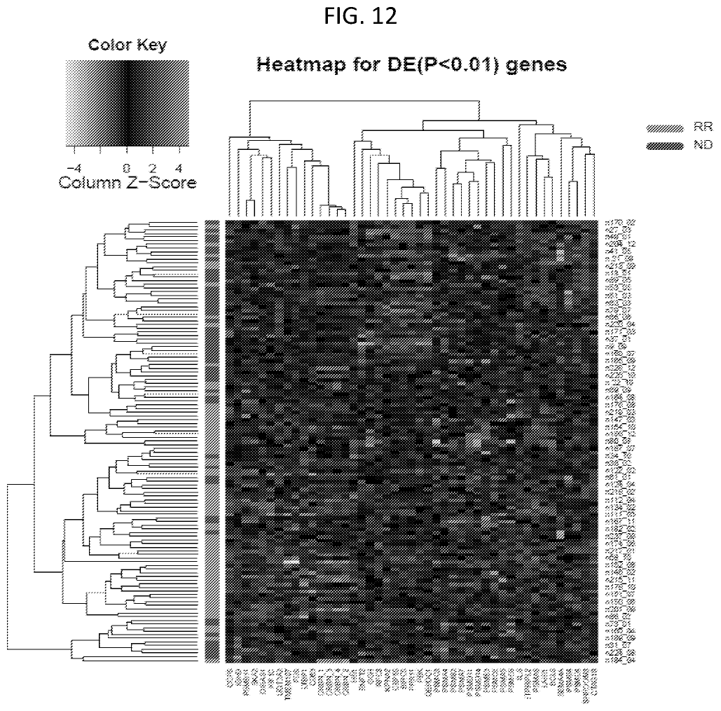

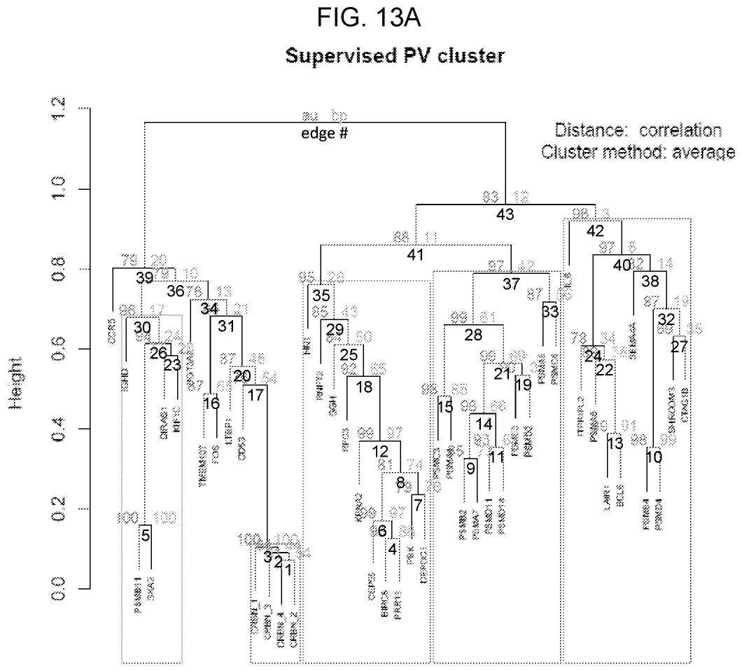

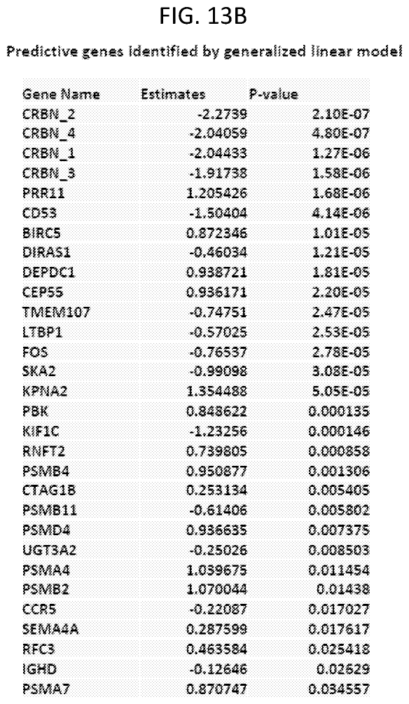

Using the NanoString profiling data obtained from all ND (n=52) and late/RR patients (n=69), the predictive value of each differentially expressed gene for drug resistance and disease progression was evaluated. In particular, edgeR software was used to identify 45/121 differentially expressed genes between ND and late/RR samples (p<0.01) (FIG. 12). The correlation of those differentiated genes was identified by clustering analysis. For example, the expression of PRR11 was found to cluster together with the expression of BIRC5, CEP55, PBK, and DEPDC1 (FIG. 13A). In addition, 31/45 genes were identified as significant predictors of disease stage (p<0.05, FIG. 13B), with CRBN, PRR11, CD53, BIRC5, DIRAS1, DEPDC1, and CEP55 being the most differentially expressed. Finally, using R-package BhGLM, a multivariable ordinal model was built that contains seven associated predictors, CRBN, CEP55, DIRAS1, SKA2, CD53, PSMA7 and PSMD14 (FIG. 14A). This model represents four IMiD sensitivity candidate genes (CRBN, CEP55, DIRAS1, and SKA2) and three PI sensitivity candidate genes (CD53, PSMA7, and PSMD14). The performance of this model was evaluated by 5-fold cross-validation with an AUC=0.91 (FIG. 14B). Using the model, RNAseq data from MMRF CoMMpass (n=578) and the Mayo Clinic MM patient dataset (n=487) also were analyzed, revealing that estimations resulting from these analyses were correlated with OS (CoMMpass data, FIG. 14C) and disease stage and treatment (Mayo Clinic MM patient data, FIGS. 14D and 14E). In addition, it was observed that samples classified toward responders through the prediction model were enriched with the samples that had a longer OS, ND samples, and samples without treatment or with fewer previous treatments.

Taken together, the studies described herein identified a subset of genes whose transcription expression was associated with IMiD/PI resistance during disease progression, and also was associated with poor clinical outcome. A NanoString gene expression profiling based prediction model for IMiD/PI resistance and disease progression also was developed; this provides a useful tool for clinical investigation and therapy selection.

TABLE 1
Probe information

| Category | Number | Gene ID |
|---|---|---|
| CRBN and IMiD related | 14 | CRBN (4), IKZF1, IKZF3, MYC, IRF4, KPNA2, ETV4, IL6, STAT3, BSG |
| | | and ZFP91 |
| Genes reported to associate with IMiD and | 24 | EMC9, FAM171B, FLNB, KIF1C, MYO9B, RCN3 and PLEK¹, ACOXL, |
| PI sensitivity | | NAP1L5, CLEC2B, RNASE6, CLIP4, SHROOM3, FRK, TCF7, IGHD, |
| | | UGT3A2 and ITPRIPL2², CD53, CCND1, FOS, LTBP1, JUN and AIM2³ |
| Genes differentially expressed between IMiD | 31 | 21 genes selected from Pomalidomide trial data and XG1/XG1res |
| and PI non-responders and responders | | analysis: BIRC5, CD52, CEP55, DEPDC1, GGH, HN1, MLLT11, PBK, |
| | | PRR11, RFC3, RNFT2, SKA2, TEX14, TMEM107, FNBP1, IFITM1, |
| | | IGKC, PTPRK, TEX9, TSPAN7 and XAF1 |
| | | 10 genes selected from MMRF coMMpass data analysis: PAX5, |
| | | ALDH1A3, ANXA1, ITGB7, LAIR1, CCR5, MYB, NRN1, TNFRSF13C |
| | | and TLR4 |
| Proteasome subunits | 40 | ALL A, B, C, D proteasome subunits |
| Genes differentially expressed in cell lines | 12 | CHRNA6, DIRAS1, NMRAL1, SEMA4A, RPS3AP29, POMT1, |
| that have variable CRBN expression level | | TBC1D16, GOLGA8S, CTAG1B, BLVRA, ARHGAP9 and FAIM3 |
| Housekeeping genes | 11 | ABCF1, G6PD, GAPDH, GPI, GUSB, MTMR14, TBP, TUBA1A, TUBB, |
| | | VCP and ZNF143 |

¹Chapman et al., Blood 132(20): 2154-2165, 2018
²Pelham et al., Blood 130(Suppl 1): 839, 2017
³Mitra et al., Leukemia 30(5): 1094-1102, 2016

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating a mammal having multiple myeloma (MM), wherein said method comprises:
(a) identifying a mammal as having a biological sample with an altered level of expression of one or more markers as compared to a level of expression of the one or more markers in a biological sample from a corresponding mammal that does not have late stage MM or refractory/resistant (RR) MM, wherein said one or more markers are selected from the group consisting of CRBN, CEP55, DIRAS1, SKA2, CD53, PSMA7 and PSMD14, thereby identifying said mammal as having or being likely to have late stage MM or RR MM, and
(b) administering to said mammal a composition comprising chimeric antigen receptor-(CAR-) T cells or a histone deacetylase (HDAC) inhibitor.

2. The method of claim 1, wherein said mammal is a human.

3. The method of claim 1, wherein said biological sample is a blood sample obtained from said mammal.

4. The method of claim 3, wherein said blood sample is a plasma sample.

5. The method of claim 1, comprising identifying said biological sample as having altered levels of expression of two or more of said markers, as compared to levels of expression of said two or more markers in said biological sample from said corresponding mammal.

6. The method of claim 1, comprising identifying said biological sample as having reduced expression of one or more markers selected from the group consisting of CRBN, DIRAS1, CD53, and SKA2, as compared to expression in said biological sample from said corresponding mammal.

7. The method of claim 1, comprising identifying said biological sample as having elevated expression of one or more markers selected from the group consisting of CEP55, PSMA7, and PSMD14, as compared to expression in said biological sample from said corresponding mammal.

8. The method of claim 1, comprising identifying said biological sample as having reduced expression of CRBN, DIRAS1, CD53, and SKA2, and elevated expression of CEP55 and PSMD14, as compared to expression of said markers in said biological sample from said corresponding mammal.

9. The method of claim 1, wherein said identifying comprises using NanoString nCounter technology to detect expression of said one or more markers in said biological sample.

10. A method for treating a mammal having MM, wherein the mammal was identified as having a biological sample with an altered level of expression of one or more markers as compared to a level of expression of the one or more markers in a biological sample from a corresponding mammal that does not have late stage MM or RR MM, wherein said one or more markers are selected from the group consisting of CRBN, CEP55, DIRAS1, SKA2, CD53, PSMA7 and PSMD14, and wherein the method comprises administering to said mammal a composition comprising CAR-T cells or a HDAC inhibitor, wherein one or more symptoms of said MM is reduced in said mammal.

11. The method of claim 10, wherein said mammal is a human.

12. The method of claim 10, wherein said biological sample is a blood sample obtained from said mammal.

13. The method of claim 12, wherein said blood sample is a plasma sample.

14. The method of claim 10, wherein said mammal was identified as having a biological sample with altered levels of expression of two or more of said markers, as compared to levels of expression of the two or more markers in said biological sample from said corresponding mammal.

15. The method of claim 10, wherein said mammal was identified as having a biological sample with reduced expression of one or more markers selected from the group consisting of CRBN, DIRAS1, CD53, and SKA2, as compared to expression in said biological sample from said corresponding mammal.

16. The method of claim 10, wherein said mammal was identified as having a biological sample with elevated expression of one or more markers selected from the group consisting of CEP55, PSMA7, and PSMD14, as compared to expression in said biological sample from said corresponding mammal.

17. The method of claim 10, wherein said mammal was identified as having a biological sample as with reduced expression of CRBN, DIRAS1, CD53, and SKA2, and elevated expression of CEP55 and PSMD14, as compared to expression of said markers in said biological sample from said corresponding mammal.

18. The method of claim 10, wherein said mammal was identified by using NanoString nCounter technology to detect expression of said one or more markers in said biological sample.
